(19) United States
(12) Patent Application Publication
(10) Pub. No.: US 2025/0110096 A1
(43) Pub. Date: Apr. 3, 2025

(54) METHOD OF QUANTIFYING POLLINATION EFFICIENCY

(71) Applicant: PowerPollen, Inc., Ames, IA (US)

(72) Inventors: Marc Albertsen, Johnston, IA (US); Jason Cope, Ankeny, IA (US); Merly Andreina Grandos de Canas, Grimes, IA (US); Federico Valverde, Polk City, IA (US); Mark E. Westgate, Ames, IA (US)

(73) Assignee: PowerPollen, Inc., Ames, IA (US)

(21) Appl. No.: 18/899,478

(22) Filed: Sep. 27, 2024

Related U.S. Application Data
(60) Provisional application No. 63/540,983, filed on Sep. 28, 2023.

(51) Int. Cl. G01N 33/00; A01H 1/02; G01N 15/06; G01N 15/075

(52) U.S. Cl. CPC G01N 33/0098; A01H 1/02; G01N 15/0606; G01N 15/075

(57) ABSTRACT

This invention describes a new method of quantifying pollen collected from floral stigmatic surfaces to quantify pollination efficiency, predict seed or fruit set, and to make further production decisions based upon the predicted seed or fruit set. The invention also describes a new method of quantifying pollen contamination on stigmatic surfaces. In addition, the method describes ways of using the pollination efficiency to make one or more evaluations of methods, treatments, equipment, or other factors. More specifically, this invention relates to a method of predicting seed or fruit set early in the crop growth cycle, enabling decisions regarding additional intentional pollinations that may be necessary in order to maximize seed, grain, or fruit set and yield.

### CROSS REFERENCE TO RELATED APPLICATIONS

This application claims priority from U.S. Provisional Patent Application No. 63/540,983 filed Sep. 28, 2023 and titled Method of Quantifying Pollination Efficiency. The entire contents of U.S. Provisional Patent Application No. 63/540,983 are hereby incorporated by reference.

### FIELD OF THE INVENTION

This invention relates generally to a novel method of quantifying pollen collected from floral stigmatic surfaces to quantify pollination efficiency, predict seed or fruit set, and to make further production decisions based upon the predicted seed or fruit set. In addition, this invention relates to a method of measuring pollen contamination by undesirable pollen present in the growing environment. More specifically, this invention relates to a method of predicting seed or fruit set early in the crop growth cycle, enabling decisions regarding additional intentional pollinations.

### BACKGROUND

The current invention has application to the field of crop seed production and grain production practices, including, but not limited to agricultural row crops, such as maize (also called corn), soybeans, wheat, rice, sunflower, canola, sorghum, cotton, barley, pearl millet, alfalfa, and other plants. The invention also has application to agricultural tree crops, such as apples, stone fruits, citrus fruits, and nuts. In addition, the invention has application to common ornamental crops, such as petunias, and impatiens. The invention can be applied to any plant species that relies on pollination for seed or grain production, including self-pollination, insect-based pollination, wind-based pollination, intentional pollination, and any other form of pollination. Specifically, the current invention provides a method for rapidly and accurately predicting seed or fruit set in pollinated crops. The current invention also provides a method for rapidly and accurately determining the level of contaminating pollen in a growing environment.

This invention is useful in all crops that use pollen to generate seed and grain, including crops that use hybrid production. Practice of this invention helps support an accurate early estimate of seed set. In addition, the invention is useful in all crops that use pollen to generate fruit, including fruit trees and crops that produce fruits, such as tomatoes, melons, cucurbits, berries, and other similar crop species. Practice of the invention helps support an accurate early estimate of fruit set. Timely delivery of sufficient pollen to ensure seed set is the primary biological factor limiting seed production, and especially hybrid seed production. Evidence from several plant species indicates more than one pollen grain per stigma is required to ensure fertilization occurs after pollination. For example, in indica rice, Satake and Yoshida (1978) determined that more than 10 pollen grains must germinate on the rice plant stigmata to ensure successful fertilization of a floret (Japanese Journal of Crop Science 47: pp. 6-10). Likewise, Sorokina and Laptev (1957) identified that on male sterile wheat plants, 4.4 pollen grains per stigma were required for 51.8% seed set (Pl. Breed. Abstr. 28, 1958:1517). More recent research has shown that in maize fields, at least 4 pollen grains per stigma were required for seed set (Westgate, Lizaso, and Batchelor. 2003. Crop Science 43:934-942). Therefore, quantitative knowledge of pollination efficiency (pollen grains/stigma) is critical for advancing natural, manual, or mechanical pollination systems for hybrid seed production for which pollen is often a limiting resource.

While methods to determine the number of pollen grains per stigma required for seed or fruit set are known, those known in the art generally involve time-consuming tissue dissection, tissue staining, and microscope analyses. There are no known existing studies that quantify pollination efficiency for commercial purposes, particularly on a row-by-row or plant-by-plant basis. As a result, companies that rely on the sale of seed for generating revenue are forced to wait until harvest to quantify the total seed volume they have available for each product. In some cases, seed shortfalls require these companies to take additional steps to generate more seed in a different global hemisphere. This step adds extensive expense, but is required to have the sales inventory to which they have committed. There is a need for faster, easier, more accurate methods to quantify pollen on stigmatic surfaces, wherein the methods do not rely on the use of stains or microscopy.

In addition, there is a need for a fast, easy method to determine contamination levels of undesirable pollen present in a growing environment before any desirable pollen is being shed. Measuring contamination can help predict genetic purity in hybrid crops, such as in seed production scenarios, but can also help predict whether a given crop will be saleable. Measuring contamination in seed production settings is a measure of seed purity. If early indications of contaminating pollen are high, the producer may elect to cease managing a given field or growing environment because the level of purity will be too low to make it financially viable to continue crop management. Similarly, if contaminating pollen is present in a field that no longer has actively shedding pollen from the desired male genetics, it can be inferred that the field is prone to a certain percent of contamination. In reference to hybrid seed, genetic purity is the measure of seeds that have the intended genetic cross between the male (also called the pollen parent) and female (also called the seed parent) parent plants, and is typically expressed as a percentage. Seeds that result from self-pollination (selfs) or pollination with unintended pollen (out-crosses) are considered contaminants and are not hybrid seeds. Hybrid corn seed must typically have 95% genetic purity (i.e. 95% of the desired genetics for the hybrid seed) to be sold as certified hybrid seed. Seed certification in the United States is a four-generation scheme that is the responsibility of each individual state, and within each state, there is an agency designated to certify seed based on the seed law of the individual state. The four generations are breeder seed, foundation seed, registered seed, and certified seed. Certified seed is produced from foundation or registered seed and is the final product of the four-generation seed certification program. Although each state has its own seed law, 95% genetic purity requirements are commonplace, with many seed companies actually adhering to genetic purity standards above 95%. Even when recommended isolation distances are used, it is common for undesirable pollen to contaminate the field due to pollen being carried by the wind, insects, and other factors of nature, from other fields located within close proximity.

Methods to separate pollen from stigmatic surfaces or other floral debris have been described. For example, Kakui et al. (2020, Plant Methods 16:124) report a filtering sequence to isolate pollen of Japanese cedar from floral debris prior to floral anthesis. In the described method, the pollen is suspended in distilled water during filtration, and then transferred to a solution compatible with a specific cell counter. This approach would not work on recalcitrant pollen species, including most grasses and various other important economic crops, because most of the pollen would burst in the water, eliminating the ability to do any meaningful pollen counts. Furthermore, the Kakui study does not use or suggest using the pollen counts for further analyses.

There is a need in the industry for technologies and methods that allow for early prediction of seed and fruit set and yield across a population of plants, the results of which enable growers to make decisions about additional intentional pollinations, even on a row-by-row prescription basis, or to make other treatments to improve seed and fruit set at a very early stage. In particular, the invention described herein provides growers with information that has a direct impact on forecasting harvesting logistics, product market value, and sales strategy.

### BRIEF DESCRIPTIONS OF THE FIGURES

FIG. 1 is a box plot comparing counts of pollen grains obtained from bouquets of maize silks pollinated with the same dose of pollen on Day 0 (left) or on Day 0 and again on Day 2 (right).

FIG. 2 is a dosage response plot showing pollen counts taken from 122 individual or bulked silk bouquets collected from multiple field experiments. The silk sample size is plotted on the x-axis and the number of pollen grains per silk is plotted on the y-axis.

FIG. 3 is a chart showing the mean pollen grain count±standard deviation across 6 wheat spikes across different treatments, demonstrating that the wheat stigmas retained a similar number of pollen grains (about 900 to 1000 grains per stigma) regardless of whether pollen was delivered in a liquid medium, dry medium, or as pure pollen.

FIG. 4 is a line chart plotting the increase in pollen collected per ear as the application dosage of pollen (in liters per acre) increased. The data are the mean±standard error for three treatment reps having four samples of five ears each.

FIG. 5 is a line chart plotting the number of pollen grains collected per gram of fresh weight of silk tissue against the application dosage of pollen in liters per acre. The data are the mean±standard error of three treatment reps having four samples from 5 plants each.

FIG. 6 is a line chart plotting the relationship between the pollen dosage per ear and the resulting number of kernels per ear, which demonstrates the pollination efficiency.

FIG. 7 is a chart showing the relationship between four treatments of different pollen application dosages and the resulting count of pollen grains collected from silk bouquets. Data are combined for the two experiments described in Example 8, and are the mean±95% confidence interval of 6 replicates of 10 ears each.

FIG. 8 is a chart showing the relationship between the number of pollen grains retained on the silk bouquet of each ear, as shown on the x-axis, and the resulting kernel set on the same ears, as shown on the y-axis. Each data point is the mean of 10 ears mechanically pollinated at dosages ranging from 2 to 8 L/ac.

FIG. 9 is a box-plot representation of the efficiency of kernel set pooled for each level of mechanical pollen dosage. The efficiencies are shown as kernels per pollen grain on the y-axis and as pollen grains per kernel in the inset, while the pollen dosages are shown in L/ac on the x-axis. Efficiency values are estimated from the mean values of kernels/pollen applied (circles in the box plot) for each application dosage.

FIG. 10 is a chart compiling data from a wide range of experiments. It shows the amount of pollen captured by silk bouquets on the x-axis and the resulting number of kernels produced by maize inbred plants on the y-axis.

FIG. 11 is a logarithmic model developed to predict kernel yield per ear of corn from pollen grains collected by receptive stigmas (silks) prior to 50% silking for the plant population.

FIG. 12 is a graph showing pollen count on receptive exposed stigmas (silks) of four different female target parents (F1, F2, F3, F4) resulting from pollinations using two different pollen donors (PD1, PD2).

FIG. 13 is a graph showing kernels per ear of four different female target parents (F1, F2, F3, F4) resulting from pollinations using two different pollen donors (PD1, PD2).

FIG. 14 is a graph of pollen counts per ear for 10 rows of female parent plants bordered on each side by one row of male pollen donor plants. The x-axis shows the two male rows as Male-East and Male-West, and ten female rows as F1 through F10. The y-axis shows pollen counts per ear. The lower curve shows average pollen counts per ear per row for naturally pollinated plants (Natural PC). The upper curve shows average pollen counts per ear for naturally pollinated plants that received an intentional commercial pollen application (Nat+Commercial PC).

FIG. 15 is a graph presenting comparing the actual and predicted kernels per ear for plants in the same ten female parent rows and bordering pollen donor rows in FIG. 14. The four curves are average kernel counts (KC) per ear measured at harvest for the naturally-pollinated plants (Natural Actual KC), the average kernel counts per ear predicted from pollen counts (PC) for naturally-pollinated plants (Natural Prediction PC to KC), the average kernel counts per ear measured at harvest for the natural+commercially-pollinated plants (Natural+Commercial KC), and the kernel counts per ear predicted from pollen counts for naturally+commercially-pollinated plants (Natural+Commercial Prediction PC to KC) for each of the male and female rows.

FIG. 16 is a processed image of maize pollen in one well of a 24-well plate prior to counting.

FIG. 17 shows a comparison of pollen counts obtained using flow cytometry (Amphasys) versus counts using an Echo Revolution fluorescent microscope and ImagePro counting software. The x-axis shows the count using Amphasys, while the y-axis shows the count using the microscope and ImagePro analysis software.

FIG. 18 is a combined bar and line chart showing measured and predicted kernels per ear for eight commercial hybrid maize seed production fields. The bars show the measured average kernels per ear at harvest maturity. The dashed line (——) shows the predicted kernel per ear values for each field using the predictive pollen presence and yield model described in this invention determined prior to 50% silking for the female parent population. The solid line (−) is the linear correlation between measured and predicted values (y=24.631x+111.54, R²=0.8531).

### SUMMARY OF THE INVENTION

Provided is a method of quantifying pollination efficiency on the basis of pollen grains per stigma, comprising growing a parent plant, waiting for the stigmatic surfaces of said parent plant to become exposed to pollen and also to become receptive to pollen, intentionally pollinating said parent plant's stigmatic surfaces with pollen collected from a pollen parent plant, and then following the hydration of the applied pollen and the entry of the pollen tube into the stigma, removing the pollinated stigmatic surfaces of said parent plant, counting the number of pollen grains retained on the stigmatic surfaces of said parent plant, and calculating the pollination efficiency for the intentional pollination. The method optionally includes predicting the parent plant's seed or fruit set based on the pollen grain count result.

The invention includes the use of the calculated pollination efficiency in different kinds of evaluations, including an evaluation of the efficiency or efficacy of an intentional pollination method; an evaluation of the efficiency or efficacy of equipment used to conduct intentional pollinations; an evaluation to determine how much additional pollen should be intentionally applied to a plurality of plants in order to maximize seed or fruit set; an evaluation to determine whether less pollen can be used in intentional pollination to successfully maximize seed or fruit set; an evaluation of a treatment used on a plant wherein the treatment is intended to enhance successful pollination; an evaluation of a treatment used on pollen to be intentionally applied to a plant wherein the treatment is intended to enhance pollen viability; an evaluation of a treatment used on pollen to be intentionally applied to a plant wherein the treatment is intended to enhance or reduce pollen adhesion to stigmatic surfaces; and an evaluation of a treatment used on pollen to be intentionally applied to a plant wherein the treatment is intended to increase pollination success.

Optionally, the provided method can include a determination of whether the predicted seed or fruit set is near maximum seed or fruit set for the given plant species. If the predicted seed or fruit set is below maximum, the method enables determining the necessary pollen grains/stigma dosage rate to achieve maximum seed or fruit set; and conducting one or more intentional pollinations at the calculated dosage rate to achieve maximum seed or fruit set.

Optionally, the provided method may include allowing the parent plant to mature and produce seed or fruit, collecting and counting the seed or fruit, and calculating the seed per pollen grain or fruit per pollen grain ratio.

The method includes embodiments wherein the removal of the pollinated stigmatic surfaces from said parent plant does not impact the successful fertilization of the plant ovule. Furthermore, the method includes embodiments in which the removal of said pollinated stigmatic surfaces from said parent plant occurs during a time period from 15 minutes following said intentional pollination to 7 days following said intentional pollination. Optionally, all stigmatic surfaces may be removed from the parent plant.

Also provided is a method of quantifying pollination efficiency comprising growing a plurality of plants, waiting for the stigmatic surfaces of said parent plants to become receptive to pollen and exposed to pollen, allowing the plants to be naturally pollinated, and following the pollination but while the stigma is still receptive, removing said pollinated stigmatic surfaces of at least one of the plants, counting the number of pollen grains retained on the removed stigmatic surfaces of the plant, and calculating the pollination efficiency for the natural pollination. The method optionally includes predicting the parent plant's seed or fruit set based on the pollen grain count result.

Further provided is a method of quantifying pollination efficiency comprising growing a plurality of plants, waiting for the stigmatic surfaces of said parent plants to become exposed to pollen and also to become receptive to pollen, allowing the plants to be naturally pollinated, following the natural pollination with an intentional pollination using pollen collected from a pollen parent plant, and then following the pollinations but while the stigma is still receptive, removing said pollinated stigmatic surfaces of at least one of the plants, counting the number of pollen grains retained on the removed stigmatic surfaces of the plant, and calculating the pollination efficiency for the combined natural and intentional pollinations. The method optionally includes predicting the parent plant's seed or fruit set based on the pollen grain count result.

The method includes various methods for counting pollen grains removed from the stigmatic surfaces, such as, but not limited to, impedance flow cytometry, densitometry, fluorescence imaging, digital imaging, cytometry, spectrophotometry, densitometry, vital dyes, hemocytometry, and brightfield microscopy.

The pollen used in the intentional pollinations of the embodiments may be fresh or preserved pollen that has been previously collected from pollen parent plants. The fresh pollen may have been harvested from one or more of a field, a growth chamber, a greenhouse, a glasshouse, a shade house, a hoop house, a vertical farming facility or a hydroponic facility. The preserved pollen may be pollen which has been previously collected and preserved by cooling, chilling, cryopreservation, freezing, freeze drying, or storage in liquid nitrogen. In addition, the pollen may have been stored as pure pollen, or may have been mixed with a solid particulate or liquid medium to aid in maintaining viability during storage. The pollen may have been collected from sources with altered circadian rhythms, sources with normal circadian flowering but wherein the male components of the parent plants are delayed, or sources with normal circadian flowering wherein said male components of said parent plants are allowed to shed with no delay. The pollen may have been obtained from single or multiple genetic sources and may have been combined before application. In addition, the pollen may be from plants with similar or different genetic backgrounds to the plants upon which it is being applied.

The pollen used in the intentional pollinations of the embodiments may be applied at the time which the parent plant first becomes receptive to said pollen. The intentional pollinations may be conducted by any one or more of: mechanical means, pneumatic means, positive pressure, negative pressure, manual means, or combinations thereof. In addition, the pollen may be applied using automated or semi-automated means, including a vehicle or drone.

The plants may be plants that comprise both female and male components, or they may comprise only female components, or may have both female and male components but the male components are not fully functional. The female components may not be covered to prevent self- or sib-pollination. In addition, the parent plants subjected to the intentional pollinations may be intended for seed production, fruit production, or grain production purposes.

In another embodiment of the invention, the method allows for the quantification of contaminating pollen presence. The method comprises growing a parent plant, waiting for the stigmatic surfaces of said parent plant to become receptive to pollen and also exposed to pollen, and then, before any desirable pollen in the growing environment begins to shed, allowing the plants to be naturally pollinated, and following the pollen hydration and entry of the pollen tube into the stigma, removing said pollinated stigmatic surfaces of the parent plant, counting the number of pollen grains retained on the removed stigmatic surfaces of the plant, and calculating the amount of contaminating pollen present.

### DETAILED DESCRIPTION

The following detailed description outlines embodiments of methods of quantifying pollen collected from floral stigmatic surfaces. These methods allow for the early and accurate prediction of seed and fruit set, which thereby enable additional production decisions. For the purposes of this disclosure, the term “prediction” means a seed set or fruit set calculation based upon the number of expected pollinations relative to the total number of potential pollinations in a reproductive environment. The resulting calculation is used to determine the opportunity to enable additional successful pollinations by conducting one or more intentional pollinations with a level of precision that may require a different quantity of pollen to be added on a row-by-row basis. The resulting calculation is also used to determine expected product availability prior to seed or fruit maturity. Such technology and methods may be used for the estimation of seed and fruit set and subsequent production decisions in the culture of any plants. Furthermore, the methods allow for the quantification of contaminating, undesirable pollen present in a growing environment before desirable pollen is being shed or applied. For the purposes of this disclosure, the quantification of contamination is the determination of the percentage of seed or fruit which is likely to result from an undesirable pollination event.

For ease of discussion and understanding, the following detailed description often refers to the invention for use with maize (also referred to as corn) or wheat, which are economically important monocotyledonous row crops. The invention can be used equally well with plants that produce fruit upon successful pollination, such as fruit trees, berry-bearing plants, and other crops that produce fruits such as cucurbits, melons, tomatoes, and similar plants. Working examples are presented using wide range of species of monocot and dicot plants. It should be appreciated that the technology and methods may be used with any pollen-producing plants, and specifically named plants, are discussed for illustration purposes only and are not intended to be limiting.

For the purposes of this disclosure, “undesirable” or “undesired” pollen is biologically compatible pollen that would pollinate the target parent plant, but which would not result in a desirable outcome. Such pollen can also be referred to as “contaminating” pollen. For example, in hybrid seed production, the desired pollen will be the one pollen that results in the targeted hybrid cross intended by the producer. Undesirable or contaminating pollen in such a situation would be any biologically compatible pollen that would successfully pollinate the parent plant but would result in incorrect hybrid seed forming, or self/sib-pollen, which would result in a non-hybrid seed outcome. An undesirable pollination event is a successful pollination of a plant's receptive stigmatic surface by pollen other than the intended pollen. The intended pollen may be pollen from a different genetic background (for cross-pollination) or from the same genetic background (for self- and/or sib-pollination).

Seed, including hybrid seed, is produced for a number of purposes. First, seed is produced for various research purposes to evaluate the value of new combinations of genetics. Seed companies devote billions of dollars to research in the pursuit of developing better plant genetics to improve hybrid seed quality. Another reason to produce hybrid seed is for the commercial sale of such seed to producers, such as farmers. In addition, seed is produced to increase the parental lines used in a hybrid seed production field. Specifically, seed in this context is intended to be planted to produce new plants. For the purpose of this disclosure, the term “seed set” means the percentage of seeds formed resulting from pollination (pollination may be either natural, intentional, or natural combined with intentional) relative to the number of fertile receptive florets that are available for pollination. To determine seed set, the following calculation is used: divide the number of seeds formed by the number of mature, receptive florets pollinated. Depending on the intended application, seed set can be calculated per floret, per whole reproductive structure (rachis, spike, panicle, etc), per plant, or per plant population. This value is typically less than “maximum seed set,” which assumes all receptive florets are available for pollination and will be fertilized to form a seed after being pollinated. Likewise, the value of ‘seed set’ is generally much less than ‘potential seed set’ which includes all developing florets whether mature, fertile, or receptive to pollen at the time of pollination. In a similar manner, the term “fruit set” means the percentage of fruits formed resulting from pollination (pollination may be either natural, intentional, or natural combined with intentional) relative to the number of fertile receptive florets that are available for pollination. To determine fruit set, the following calculation is used: divide the number of seeds formed by the number of mature, receptive florets pollinated. Depending on the intended application, seed set can be calculated per floret, per whole reproductive structure (rachis, spike, panicle, etc), per plant, or per plant population. In cases where the floret ovary contains many ovules and resulting fruit structures can produce many seeds (i.e. aggregate fruit; e.g. berry, pepo, hesperidium, legume pod), “fruit set” is distinguishable from “seed set” by counting the number of seeds per fruit.

For the purposes of this disclosure, the maximum seed or fruit set is the percentage of seeds or fruits formed resulting from pollination (pollination may be either natural, intentional, or natural combined with intentional) relative to the number of fertile receptive florets available for pollination assuming all receptive florets available for pollination are pollinated and form a seed or seed-bearing fruit once pollinated. Depending on the intended application, maximum seed and fruit set can be calculated per floret, per whole reproductive structure, per plant, per row, or per plant population. In species where the floret ovary contains many ovules and resulting fruit structures can produce many seeds, “maximum fruit set” is distinguishable from “maximum seed set” by counting the number of seeds per fruit. The maximum seed or fruit set is distinguishable from the “potential” seed or fruit set. The potential seed or fruit set is the number of developing florets, whether mature, fertile, or receptive to pollen, independent of pollination potential. The predicted seed or fruit set can be calculated for a given plant species by developing a logarithmic model. This is further detailed in working example 15 of this disclosure.

Grain, in contrast, is produced for a number of purposes, including human consumption, animal consumption, industrial use, and for research purposes. The primary goal of grain production is to harvest the largest amount of a product having the greatest market value. Seed companies devote billions of dollars to research in the pursuit of developing better plant genetics to improve grain yields and grain market value. Regardless of the end use of the grain, the production of grain is dependent on the appropriate pollen fertilizing the appropriate plant at the appropriate time. Specifically, in this context, the term “grain” excludes production of seed that is intended to produce new plants. The term “industrial use” is intended to mean uses in which the grain is destroyed or modified so that it cannot be planted to produce new plants. Examples of industrial use include but are not limited to ethanol production, biofuel production, production of flour, industrial powders, other foods for human or animal consumption, production of oils, and similar industrial processes and modifications that render the grain non-fertile and non-viable.

The term “grain” is also used herein to describe pollen. A “pollen grain” is a microscopic plant structure that carries the male reproductive cells of the plant. Each pollen grain contains a tube cell (containing the nucleus that controls formation of the pollen tube) and a generative cell (containing the nucleus that divides into two sperm nuclei). Once in contact with the stigma (receptive structure of a female flower), the pollen grain produces a pollen tube that carries the sperm nuclei to the ovule. Fertilization occurs when the sperm nuclei combine with separate nuclei in the ovule to form the embryo and endosperm.

Fruit is likewise produced for human and animal consumption, for industrial use, and for research purposes. The primary goal of fruit production is to harvest the maximum amount of fruit having the highest market value. Regardless of the end use of the fruit, the production of fruit is dependent on the appropriate pollen fertilizing the appropriate flower at the appropriate time. The term “industrial use” is intended to mean uses in which the fruit is destroyed or modified. Examples of industrial use include but are not limited to ethanol production, biofuel production, production of fruit juices, fruit purees, fruit-based food products, fruit powders, other fruit-based foods for human or animal consumption, production of oils, and similar industrial processes and modifications. The seeds located within the fruit may potentially be used for other purposes, including planting or for industrial uses in which those seeds are destroyed or rendered non-viable.

This invention can be used during the production of any hybrid or non-hybrid seed, grain, or fruit produced for any purpose. Regardless of the end use of the seed, grain, or fruit, its production is dependent on the appropriate pollen fertilizing the appropriate flower at the appropriate time such that sufficient pollen lands on the stigmatic surface to ensure seed or fruit set. The invention can be used in situations where natural pollination is occurring, such as insect- or wind-mediated pollination, or with self-pollination. In addition, this invention can be used with intentional pollination methods. In such methods, pollen is collected, optionally stored, and subsequently intentionally delivered to plants. For example, U.S. Pat. No. 4,922,651 discloses an apparatus for effecting or improving pollination of plants. U.S. Pat. Nos. 10,905,060 and 10,398,099 describe intentional pollination for seed production and grain production, respectively.

Use of the term “intentional pollination” with regard to pollen application means the specific application of pollen onto receptive stigmas in a way that does not include natural pollination by wind, insect activity or other naturally occurring conditions. Intentionally applied pollen is pollen that has been deliberately applied to a plant as a result of a deliberate human activity, decision, or intervention, and may be applied by hand or by other means, such as mechanized delivery. Intentional pollination can be conducted in any number of ways, including, but not limited to, manual delivery, manual delivery with a small hand mechanical device for semi-automated dispersal, by field driven machinery containing pollen dispersal machinery or via fully automated dispersal by a self-propelled and/or human guided apparatus such as a drone that has a pollen dispersal device mounted to it, wherein the pollen dispersal is by automatic means, including, but not limited to, mechanical or pneumatic means. Use of the term “intentional pollination” excludes the deliberate planting of plants in close proximity to enable wind- or insect-mediated pollination, which for the purpose of this application is considered to be natural pollination and not intentional pollination. The term “natural pollination” means pollinations resulting from pollen released from male flowers and delivered to receptive stigmas by natural means such as by wind, bees, other insects, or animals without regard to the proximity of male flowers producing pollen to the female flowers bearing receptive stigmas.

For the purposes of this disclosure, the term “pollination efficiency” means the percentage of pollinations (either natural or intentional) that are successful, based on the number of fertile stigmas available during pollination. For the purposes of this disclosure, the term “pollination efficacy” means the number of pollen grains per stigma required to ensure fertilization of the floral ovary or ovaries. For the purposes of this disclosure, the term “pollination intensity” means the quantity of pollen in an environment delivered to female flowers naturally or intentionally during the period of stigmatic receptivity. Intensity can be quantified per unit area, per unit time, or per flower. For the purposes of this disclosure, the term “stigmatic receptivity” means the physiological status of the pollen receptive surface (stigma) of a pistillate (female) flower relative to its capacity to support successful pollination and fertilization of the ovary. A receptive stigma promotes pollen capture, pollen adhesion to the stigma surface, pollen hydration required for germination, pollen tube entry into the stigma body, and growth of the pollen tube within the style. As recognized by one of skill in the art, depending on the species of plant, the hydration and dormancy status of the pollen, and environmental conditions, these processes generally take at least 15 minutes from the time pollen contacts the stigmatic surface. Variation in stigma receptivity to pollen involves a complex interplay of genetic compatibility, stigma surface biochemistry, and environmental effects on stigma development and physiology.

This invention can operate in any crop plant that relies on pollen for seed, grain, fruit, or vegetable development. It can operate in any growing environment including, but not limited to, ideal or target growing environments, off-season environments, or controlled environments (e.g., shade/glass/green/hoop houses, growth chambers, vertical farming facilities, hydroponic facilities, aeroponic facilities, etc.).

For the purposes of intentional pollination, the acquisition of pollen can be from any source of freshly collected or previously collected and stored pollen, including pollen that has been collected from one or more pollen sources. The pollen must have been stored in such a way that the pollen retains sufficient viability to enable desired seed or fruit set. The plants that have been used as the pollen source for such a pollen bank may have been grown and harvested in any conditions, including but not limited to, a field, a growth chamber, a greenhouse, a glasshouse, a shade house, a hoop house, a vertical farming facility or a hydroponic facility. Pollen may also be harvested from an anther studio, which enables optimal growth conditions for plant reproductive tissues for any species or variety of plant. Plant reproductive tissues (corn tassels for example) are cut from plants growing in standard outdoor conditions, such as in the field or those grown in controlled conditions, such as the greenhouse or a growth chamber. The tissues are preferably cut prior to the plant beginning to shed pollen and are placed into the anther studio. The tissue may then be cultured in a nutrient medium allowing for further growth. Preserved pollen may have been preserved by any means that permits the pollen to retain viability, including but not limited to various forms of cooling or freezing including, but not limited to, chilling, cryopreservation, freeze drying, or storage in liquid nitrogen. Pollen can also be subjected to field conditioning and preservation as described in U.S. Pat. Nos. 10,575,517, 11,344,027, and US Patent Application Publication US20190008144.

Intentional pollinations can occur as soon as target parent plants are receptive to pollen. Alternatively, or in addition, intentional pollination may occur at any other time during the receptivity of the stigmatic surface. In other words, the intentional pollination can occur at any time that the female component of the plant is open to receive pollen. In different plant species, pollen receptivity occurs for different periods of time. For example, in corn, female components are receptive to pollen prior to emergence of the silks and remain receptive to pollen for 2 to 7 days, or in some case more than 7 days after emergence, depending on genetics and environmental conditions. The duration of floral receptivity to pollen varies widely across plant species from minutes to many days (Heslop-Harrison, J. S. (1992). The Angiosperm Stigma. In: Cresti, M., Tiezzi, A. (eds) Sexual Plant Reproduction. Springer, Berlin).

Accordingly, this invention can be practiced anytime during the receptivity period of the species on which the invention is being practiced, and can be practiced at any time after the stigmatic surface becomes receptive. The invention can be practiced on multiple days during the receptivity period of the stigmatic surfaces. Pollination may be allowed to proceed naturally, or pollen that has previously been collected from pollen parent plants may be intentionally applied any number of times, including but not limited to, once per day, multiple times per day, or in a continuous application. In addition, natural pollinations may be allowed to occur which are preceded or followed by intentional pollinations. A “pollen parent” plant is a plant that produces biologically compatible pollen that can successfully pollinate the target parent plant. The pollen parent plant may be a similar genetic background, such that it produces sib-pollen that is genetically the same as the target parent onto which the pollen is applied, or it may be a different genetic background, such that it produces pollen that is genetically different from the target parent onto which the pollen is applied, resulting in a cross-pollination. A “parent plant” or “target parent plant” is the recipient of the pollen from the pollen parent plant. The parent plant is a reproductively mature plant with receptive stigmatic surfaces that is ready to receive pollen. In the case of self-pollination, the parent plant and the pollen parent plant are genetically the same and may even be the same single plant. In the case of cross-pollinations, the parent plant and the pollen parent plant are genetically different.

If intentional pollinations are being conducted in the practice of the invention, the timing of pollen delivery to parent plants is important. In all crops, there is a cycle during the pollination window (the time during which the parent is receptive to pollen and during which a successful pollination event can occur) in which pollination of female flowers is most effective. This occurs because the male components of parent plants have a distinct daily cycle in which the male flowers mature and shed viable pollen from dehisced anthers. In cereal crops for example, pollen shed typically begins by mid-morning and ends by late morning or early afternoon. In dicot crops, such as cucurbits, the daily cycle coincides with the daily activity of insect pollinators. If one is intentionally applying pollen to parent plants, one may choose to apply pollen several days before pollen would typically begin to shed or at several times of the day or in a continuous fashion to increase the probability of successfully maximizing seed or fruit set. For example, pollen can be applied in accordance with the methods described in U.S. Pat. Nos. 10,905,060; 10,398,099; 11,166,422; 11,166,421, or US Patent Application Publications US20210076583; US20210259175; US20210386030; or US20210059276.

Pollen grains are small and can be very delicate. The ability of pollen grains to successfully bring about fertilization can be compromised by a range of environmental stresses. This ability also can be limited by inherent traits passed on by the plant producing the pollen. Such inherent and stress-induced impacts on pollen performance are typically described in terms of vigor, viability, and longevity. Pollen vigor generally is quantified in terms of speed of pollen germination or rate of pollen tube growth. Pollen viability often is described in terms of percent germination, but is more rigorously quantified as the ability of pollen to successfully sire viable seeds (fertilization of female egg cell and endosperm) (Shivanna, K R et al. (1991) Theor. Appl. Genet. 81 (1): 38-42). Pollen longevity, as the term implies, refers to the duration of time pollen remains viable once shed from the anthers. Pollen vigor, viability, and longevity can vary significantly among plant species, cultivars, and varieties.

Natural existing variation in pollen longevity among plant species may be further affected by environmental conditions (Dafni, A. & D. Firmage (2000) Plant Systemics and Evolution 222 (1): 113-132). Higher humidity and lower temperatures may extend pollen longevity. For example, in rice (Oryza sativa), pollen longevity has been found to be as short as 4 minutes (Koga et al. (1971) Cytologia 36:104-110) or up to 20 minutes for 50% of the pollen to lose viability (Khatum, S. and T. J. Flowers (1995) J. Exp. Bot. 46:151-154). In contrast, field grown radish (Raphanus sativas) pollen was shown to have a 5-day lifespan (Siddiqui, B. A. (1983) Acta Bot. Ind. 11:150-154).

Floral structures are initiated and develop sequentially as a natural aspect of plant reproductive development. The sequential development of a reproductive inflorescence is expressed externally in a variety of ways (e.g. rachis, panicle, corymb, spike, etc.). This sequential developmental program creates an inherent asynchrony in floral maturation (anthesis) between the earliest and latest formed flowers within an inflorescence, and between inflorescences on the same plant (Uribelarrea et al., (2002) Crop Sci., 42:1910-1918; and (2008) Field Crops Res. 105:172-181). This developmental asynchrony favors successful pollination and seed formation in the early-formed flowers at the expense of the later-formed flowers (Cárcova and Otegui (2001) Crop Sci. 41:1809-1815). In maize, for example, numerous field studies have confirmed that synchronous pollination of early and late form florets within and between inflorescences overcomes the disadvantage of late-pollinated flowers to produce seeds (Westgate et al, (2022) Crop Sci. 62:2067-2075). A similar phenomenon of floral asynchrony occurs at the plant population level in open-pollinated plants. Variability in plant development caused by the environment or plant management can create an unfavorable asynchrony between the peak of pollen shed and the peak of female floral receptivity required for maximum seed set. This phenomenon of asynchrony between pollen shed and floral receptivity to pollen is especially evident in hybrid seed production systems that use genetically distinct inbred plants as pollen donors and receptive females. Low levels of pollen production typical of hybrid seed systems greatly favor seed formation on early formed flowers on the earliest flowering plants. The impact of developmental asynchrony can be overcome, however, by an intentional application of additional pollen which promotes the synchronous pollination of all exposed and receptive florets throughout the population (see Example 21).

When a pollen grain lands on a stigmatic surface, it does not always result in a successful fertilization event. First, the pollen must be compatible with the stigma, in most cases such that both are from the same species of plant. In addition, there must not be any self-incompatibility between the pollen and stigma, which can occur in some plant species, blocking fertilization between two genetically similar gametes. If the pollen is compatible with the stigmatic surface, the pollen absorbs water from the stigma and a pollen tube begins to form, starting from the pollen grain. The tube enters the stigma and grows within the style toward the ovule. In some plants, such as some gymnosperms, pollen tube growth can be extremely slow, taking up to a year for successful fertilization. In other species, the pollen tube grows extremely quickly, at rates of 1 cm per hour. When the pollen tube reaches the ovule, it releases two male sperm cells, one of which fuses with the egg cell nucleus and the second fuses with the polar nuclei outside the egg cell resulting in a double fertilization required to form the embryo and endosperm. This is the beginning of seed or fruit development.

Accordingly, given the many factors that affect pollen viability, vigor, and longevity, when intentional pollination is being used in the practice of the invention, care must be taken to use pollen that remains viable and that is likely to result in successful development of pollen tubes and subsequent seed or fruit set.

Seed set response to natural pollen shed density is well documented. For example, Westgate, Lizaso & Batchelor (2003) studied the quantitative relationships between pollen shed density and grain yield in maize (Crop Science, 43 (3), 934-942). None of these studies, however, directly quantified effective pollen engagement on the stigma surfaces. Thus, in the literature at the time of the invention, the actual efficiency of pollination is not reported, regardless of whether it is measured by pollen per stigma, pollen per seed, or pollen per fruit. The described method recovers the pollen from pollinated stigmas, making these measures of pollination efficiency possible.

Research supporting this invention has identified that by counting the number of grains of pollen retained on stigmatic surfaces, an accurate estimate of seed or fruit set can successfully be determined. This assessment can be completed whether the pollination has occurred naturally or as a result of intentional pollination, or a combination of the two pollination methods. This assessment can be conducted very soon after pollination begins, or it can be conducted after a longer period of time during which the stigmatic surfaces remain receptive. For example, the counting can be conducted 15 minutes after pollination has occurred to allow for pollen/stigma adhesion (Taylor and Hepler (1997) Annu. Rev. Plant Physiol. Plant Mol. Biol. 48:461-491), several hours after pollination has occurred, or up to 7 days after pollination has occurred. For example, the counting can be conducted at any time after pollination has occurred, such as after 15 minutes, 30 minutes, 45 minutes, 1 hour, 2 hours, 3 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or after 1 day, 1.5 days, 2 days, 2.5 days, 3 days, 3.5 days, 4 days, 4.5 days, 5 days, 5.5 days, 6 days, 6.5 days, or 7 days after pollination. The ideal length of time will vary based on the plant species being assessed as well as environmental conditions that affect the plant, such as humidity, drought, insect pressure, temperature, and other biotic and abiotic factors. The range of time during which counting can be performed can be any range in the above-described timeframe, for example, at least 15 minutes and up to 7 days after pollination, or at least 30 minutes and up to 6.5 days after pollination, or at least 1 hour and up to 5 days after pollination. In some species with many stigmatic tissues, such as corn, it may be advantageous to allow more time for pollinations to occur, such that the count occurs at least 12 hours after pollination, and up to 4 days, or more, following any pollination.

Practice of the invention may involve the harvesting of stigma tissue. In such cases, one of skill in the art will recognize that any subsequent intentional pollinations are being conducted on the remainder of the plants in a population because the plant or plants from which stigma tissue was harvested cannot be pollinated again. In other cases, harvesting of the stigma tissue may not be required, and nondestructive methods may be used to assess the number of adhering pollen grains. Such methods may include indirect methods including fluorescence tagging, digital imaging, microscopy, and digital scanning. In addition, if the desired outcome of the assessment is to determine whether additional intentional pollination is required, the assessment must be completed while the stigmatic surface is still receptive to pollen, such that any additional pollinations occur while the stigma can still support pollen germination and pollen tube growth.

Pollen grains with compatible stigma interactions are known to adhere quickly to stigmatic surfaces through physical/chemical interactions and the attachment is reinforced by pollen tube entry into the stigma, which is an indirect indicator of pollen health and pollen-stigma compatibility. Therefore, it is possible to harvest pollen from stigmatic surfaces after pollination without loss of pollen counts. When the described method is used to quantify pollen/stigma after ovary/ovule fertilization has occurred, the user can quantify pollen/stigma and pollen/seed (or fruit) on the same set of flowers. One of skill in the art can readily determine how soon after pollination the whole stigmas or exposed and pollinated portions of the stigmas can be excised without disrupting the formation of seeds or fruit.

The pollen tube grows at the tip and is isolated from the older parts of the tube by a succession of callose plugs. The tube tip keeps growing even if older parts in the stigma/style of the female flower are removed (such as the pollinated ends of corn silks). (Qin, P. et al. (2012). BMC Plant Biology 12, Article 178). The health of silks or stigmatic tissues can be affected by the moisture status of the tissues (Herrero & Johnson (1981). Crop Sci. 21:105-110; Turc et al. (2016). New Phytol., 212:377-388)). Pollen attachment to stigmatic surfaces is influenced by moisture status, which influences pollen adhesion. Adhesion is also assisted by the presence of trichomes. In addition, the formation of the pollen tube can help maintain the adhesion. (Mól et al., (2004) Plant Sci. 166:1461-1469; Broz & Bedinger (2021). Ann. Rev. Plant Biol. 72:615-639). Work conducted as a result of this invention revealed the surprising discovery that the pollen grains remained adhered to the stigmatic surfaces for a very long period of time after attachment. In addition, it was surprising that the counting of the adhered pollen grains provided such an accurate prediction of yield, as further described in the working examples of this disclosure.

This invention is the first demonstration of a method that quantifies pollination efficiency on a pollen per stigma and seed (or fruit) per pollen grain basis. It enables seed or fruit set prediction based on pollination (natural or intentional) under favorable and unfavorable conditions. It also enables the user to distinguish pollination failures due to male gametophyte or female gametophyte limitations. For example, an increase in seed or fruit set corresponding to an increase in pollen dosage implies a limitation in availability of male gametophytes per stigma until the pollen dose is saturating for seed or fruit number. No further increase in seed or fruit set with higher pollen doses implies a female gametophyte limitation, especially if the number of seeds or fruits set is less than the number of receptive female florets. Such assessments are only possible if the number of pollen grains delivered per stigma is known.

As already mentioned, another embodiment of the present invention is a method for measuring contaminating undesirable pollen in a growing environment. This is particularly useful for assessing the level of contamination prior to the commencement of any natural pollen shed of either desirable self- and/or sib-pollen or of a specific desirable non-self pollen to achieve cross-pollination. Assessing the level of contaminating or undesirable pollen in the growing environment helps to determine how much contaminating pollen is coming from other sources, such as neighboring fields, and how this may affect any intended natural pollination or any intentional pollination using previously collected pollen. The measurement of contamination can help the producer make further crop management decisions. For example, a grower may decide that a field is experiencing contamination levels that are too high and that it is not financially viable to continue managing that crop with the intent of selling it. This is because the invention can be used to predict how much seed (or grain) would be an undesirable cross, which may be too high for the product to be sold in the intended market. In the case of cereal crops, the producer may decide to change production of that field to a grain (rather than seed) field, which could require lower inputs and save on production costs. Alternatively, the producer may decide to immediately proceed with an intentionally applied pollen treatment to overwhelm the presence of the undesirable pollen and ensure the desired cross-pollination occurs in as many instances as possible.

One of skill in the art will understand that this invention allows a grower to overcome the limitations of natural pollination. Many variables can interfere with natural pollination, such as extreme temperatures, lack of precipitation, unfavorable wind events, lack of synchrony, pollinator insect density, and other abiotic and biotic factors. This invention allows the grower to determine how well natural pollination is occurring, and then predict the seed or fruit set likely to result from the natural pollination. It enables calculation of the pollination efficiency with natural pollination, given any variables that are present. After calculating the pollination efficiency, the grower can decide whether to conduct one or more intentional pollinations to make up for any shortfall in natural pollination due to any variables that may be present and affecting natural pollination levels. So enabled, a grower can effectively overcome the negative factors affecting natural pollination and expected seed or fruit yield.

Embodiments of the invention include predicting seed or fruit set in a plant per pollen grain adhered to a stigmatic surface. The method quantifies actual numbers of pollen grains that have encountered stigmatic surfaces of flowers. Adhering pollen grains are dislodged from stigmatic surfaces by agitation into a solution for subsequent counting. One of skill in the art will be able to determine the degree of agitation required to dislodge the pollen grains without damaging them. Methods that destroy or significantly damage the pollen grains are less accurate. The pollen grains are dislodged using a brief agitation in isotonic solution or in other solutions which do not permeate the pollen membrane, and which are subsequently filtered to exclude non-pollen contaminants. One of skill in the art will be able to select an appropriate filter pore size in order to retain the pollen of the species of interest while allowing smaller particles to pass through the filter. An isotonic solution is recommended for accurate pollen counts (particularly with recalcitrant pollen species such as those from Poaceae plant species) because it limits the number of false counts resulting from burst pollen. The remaining pollen grains are then quantified by a cell counting method (e.g., impedance flow cytometry).

Depending on factors such as the plant species, number of plants, and other considerations, it may be beneficial to take a number of representative samples of stigmatic surfaces from the growing environment to assess for pollen counting, including samples taken over the course of multiple days. In some cases, all stigmatic surfaces are removed from one or more plants, while in other cases, only the pollinated portion of the stigmatic surfaces is removed. Estimates for pollination efficiency can be extrapolated from counts based on a pollinated portion of the stigmatic surfaces. In some plant species, the stigmatic tissues are not all exposed at the same time. In such cases, only the exposed portion needs to be removed because that is the portion that has been pollinated. Samples may be collected from different rows of plants in a field or different parts or areas of a field (or any other growing environment) in order to assess whether pollination efficiency is consistent across all plants and across all rows and/or areas of the growing facility. These assessments help determine areas where additional intentional pollination is required. In some cases, areas of a field or other growing environment can be affected or impacted by a variety of conditions, including weather conditions, soil conditions, insect pressure, other biotic or abiotic conditions, human-induced situations or environmentally induced situations, or any other factor that may alter the growth, development, or successful pollination of plants. By measuring natural and/or intentional pollination efficiency, an evaluation of an entire growing environment can be made and the need for additional pollinations can be determined.

This method provides a rapid, accurate, and consistent means to confirm efficacy of pollen delivery to stigmatic surfaces. It is equally effective whether the pollen has been deposited by natural means or by intentional pollination (artificial means). When coupled with measurement of the subsequent seed or fruit set, the method also provides an in vivo estimate of efficiency for the pollination process (pollen grains required/seed) for the plant species of interest. Calculating the efficiency of natural, manual, or mechanical pollen application provides, for the first time, an accurate prediction of seed or fruit set well in advance of normal seed or fruit maturation. Such information is fundamental to establishing return on investment (ROI) of any natural pollination or intentional pollen application, whether with freshly collected or preserved pollen.

The cell counting method used to quantify the pollen collected from the stigmatic surfaces can vary, and various counting methods are known in the art. Research on this invention used impedance flow cytometry with pollen in Isoton™ II diluent solution (Beckman Coulter) (hereafter referred to as Isoton). However, pollen numbers per sample can be measured by any method suitable for counting pollen grains, including impedance flow cytometry, densitometry, fluorescence imaging, digital imaging, cytometry, spectrophotometry, densitometry, vital dyes, hemocytometry, brightfield microscopy, and other image or cell counting methods.

The described invention allows for a wide range of evaluations and decisions to be made based on the calculated pollination efficiency. Such evaluations may include the assessment of efficacy, efficiency, or effectiveness. Efficacy is the capacity of producing a desired result, with an emphasis on quality and impact, while efficiency is the measurement of resource use with the intent of minimizing resources for maximum output—the most economical use of resources, such as time, effort, and materials. Effectiveness is a measure of adequacy for a given purpose. As such, the invention can measure whether a goal is achieved, how well the goal is achieved, and whether the goal is achieved in an economical fashion. The disclosed invention may also be used to refine future crop management decisions, including altering the number, position, and/or planting rate of pollen parent (male) and pollen recipient (female) plants, the timing for planting pollen parent (male) and pollen recipient (female) plants and the general layout of the male and female plants. Altering these factors based on the pollination efficiency calculations can result in improvements in return on investment for the grower. Furthermore, the invention allows for the estimation of the percent of male pollen quantity remaining during the pollination period based on the relative maturity of the male reproductive structures. The percentage of pollen shed can be predicted, and the remaining quantity of pollen can also be predicted, based upon known data pertaining to pollen biology for a given species. The counting of pollen from the receptive stigmas shows a specific ‘snapshot in time’ that allows for very accurate determination of where additional intentional pollinations are required—not just in a field in general, but by row or by area of a field.

In one embodiment, the invention allows for the evaluation of the efficiency (or efficacy (capacity of producing the desired result) of an intentional pollination method or technique. For example, different methods of intentionally applying pollen to plants, different timing of intentional application of pollen to plants, or testing intentional pollinations on previously untested species of plants, may have various levels of efficacy and result in varying levels of success. The present invention allows for the analysis of such intentional pollination methods and techniques and their expected outcomes without having to wait until the plants have fully matured and the seed or fruit set can be counted. Instead, the invention provides an early and accurate measurement technique to assess the method being used and focus on the best and most successful iterations.

Likewise, the described invention can be used to evaluate the equipment used to conduct intentional pollinations, such as pollen applicators, or specific aspects of applicators, such as nozzle types. The invention can be used to compare the results of intentional pollinations using different equipment configurations or in different conditions with the same applicator. Such evaluations will allow for the improvement of intentional pollination equipment and associated methods. Again, the invention allows the analysis of such equipment and its use to be known and compared shortly after pollinations are conducted, rather than having to wait for the plants to fully mature and set seed or fruit before the impact of using certain equipment or the modifications to equipment can be assessed and understood.

In another embodiment, the present invention allows for the determination of how much additional pollen should be applied to a plurality of plants to maximize seed or fruit set. In some cases, natural pollination may be allowed to occur, and following a period of natural pollination, the invention can be practiced to determine the natural pollination efficiency. Based on that calculation, further pollination decisions can be made. For example, it may be determined that natural pollination was sufficient (or more than sufficient) to maximize seed or fruit set. It may also be determined that additional pollination is required to maximize seed or fruit set and that therefore, one or more intentional pollinations should be conducted on the plurality of plants. It may even be determined that natural pollination was extremely poor, resulting in potential crop failure, and allowing the grower to remediate the plurality of plants by planning intentional pollinations during the remaining period of receptivity. By evaluating the efficiency of natural pollination, decisions regarding future planting can be improved. For example, a producer may decide to use a different pollen parent in future, or they may decide to increase or decrease the presence of the selected pollen parent in their fields. They may also decide to plant the pollen parent earlier or later in the following season.

Similarly, the present invention can be used to evaluate the efficiency of an intentional pollination event. If natural pollination did not occur and intentional pollination was used instead, or if natural pollination occurred but was supplemented by intentional pollination, the success of the intentional pollination can be measured by practicing the present invention. The invention can identify whether the intentional pollination event was sufficient to maximize seed or fruit set, or whether further intentional pollinations are required. Such an analysis allows for real-time decision-making by the grower to compensate for potentially poor natural pollination or intentional pollination events, thereby giving a higher likelihood of a successful crop harvest.

In another embodiment, the present invention can be used to determine how much additional pollen should be intentionally applied to a plurality of plants in order to maximize seed or fruit set. For example, in the case of a crop that has been allowed to pollinate naturally, the natural pollination level can be evaluated. It may be determined that additional pollination is necessary to maximize the seed or fruit set. One skilled in the art will recognize that different populations of plants may require different amounts of intentionally applied pollen throughout the population. Similarly, if an intentional pollination was already conducted on a plurality of plants, the invention can be used to determine whether additional pollination is necessary and if so, how much pollen should be applied. Additional intentional pollinations may be used in a variety of situations, for example, in environments which have large gaps in plant maturities across the population or in situations where environmental stressors require maximum volumes of pollen to result in maximized seed or fruit sets.

In some cases, intentional pollination may be conducted and the grower may wish to determine whether less pollen could have been used to result in the same seed or fruit set outcome. Because intentional pollination often uses pollen that may need to be stored or preserved in specific conditions, or may need to be subjected to specific treatments, the pollen used in intentional pollinations can be a limited and/or costly resource. In such situations, it is beneficial to use as little pollen as possible to maximize seed or fruit set. Practice of the invention allows for an assessment of pollination efficiency at a specific application rate. Tests can be conducted to determine whether a lower application rate would result in an equal level of seed or fruit set, thus allowing for conservation of a limited pollen resource.

In other embodiments, the present invention can be used to evaluate experimental or test treatments. The treatments may be conducted on the plant that is to be pollinated or on the pollen used in intentional pollinations. The invention allows the assessment of such treatments with the ability to demonstrate results shortly after pollination, rather than having to wait for the treated plant, or the plant pollinated with treated pollen, to mature and produce fruit or seed.

In one embodiment, the plant may be treated with a substance intended to enhance successful pollination. For example, the treatment may be intended to improve the pollen's ability to adhere to the stigmatic surface, or it may be a treatment intended to lengthen the period during which the plant remains receptive to pollen, thereby allowing more opportunity for pollination to occur. In situations such as these, the invention can be practiced to assess pollination efficiency following the treatment of the plant. The invention will provide a reliable estimate of the fruit or seed set that will result, thereby allowing assessment of the success or failure of the treatment and its intent. In contrast to the embodiment just described in which the intent of the treatment is to enhance pollination, other treatments may be intended to increase pollination success. For example, in a similar embodiment, the plant may be treated with a treatment that is intended to increase pollination success, such that more pollen grains successfully pollinate the stigmatic surfaces, thereby resulting in maximization of fruit or seed set. The invention can be practiced in such circumstances to provide a pollination efficiency measurement, thus providing an indication of whether the treatment is likely to be successful in either enhancing pollination success or increasing overall successful pollinations.

In other embodiments, the invention can be used to evaluate a treatment applied to the pollen rather than the plant. For example, pollen may be treated in ways that are intended to enhance the pollen's viability. Such a treatment would be expected to result in a higher number of successful pollinations because more of the pollen grains applied to the plant would be viable. Practice of the invention will determine the pollination efficiency and thus demonstrate whether the treatment was successful, or which treatments were more successful than others. Likewise, in a similar embodiment, a treatment may be applied to the pollen in order to enhance its adhesion to the stigmatic surfaces of the plant. Such a treatment would be intended to result in a higher number of successful intentional pollinations. The practice of the invention would allow for evaluation of pollination efficiency using the treated pollen, which could be compared to control pollen. The comparison of the results would demonstrate whether the treatment was successful.

In yet another embodiment, the described invention may be used on a plant wherein the plant has been treated to temporarily reduce pollen adhesion to the stigmatic surfaces. Such a treatment could be used to prevent natural pollinations during a time when a nearby field is releasing an undesirable but compatible pollen. Such a treatment could also be used to prevent undesirable self- or sib-pollination events. The treatment could be used to prevent such pollination events for a limited period of time such that intentional pollination could be used on the plants at a later time but still during the period of receptivity. The invention can be used to evaluate the success of the treatment and its ability to prevent undesirable pollination during a selected period of time. If the practice of the invention showed a high pollination efficiency, it would demonstrate the poor performance of the treatment, whereas if the practice of the invention showed a poor pollination efficiency, it would confirm the effectiveness of the treatment.

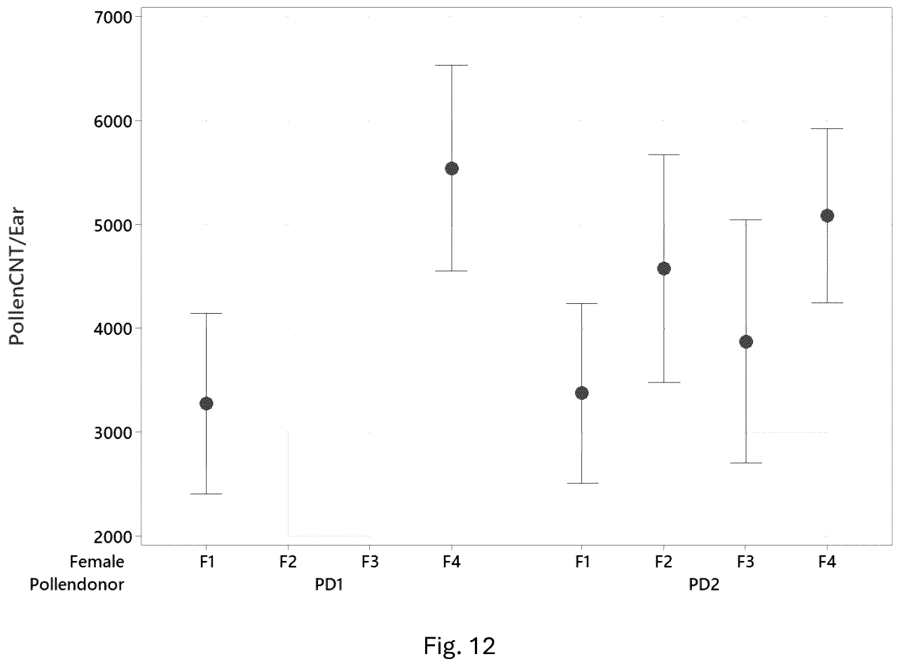

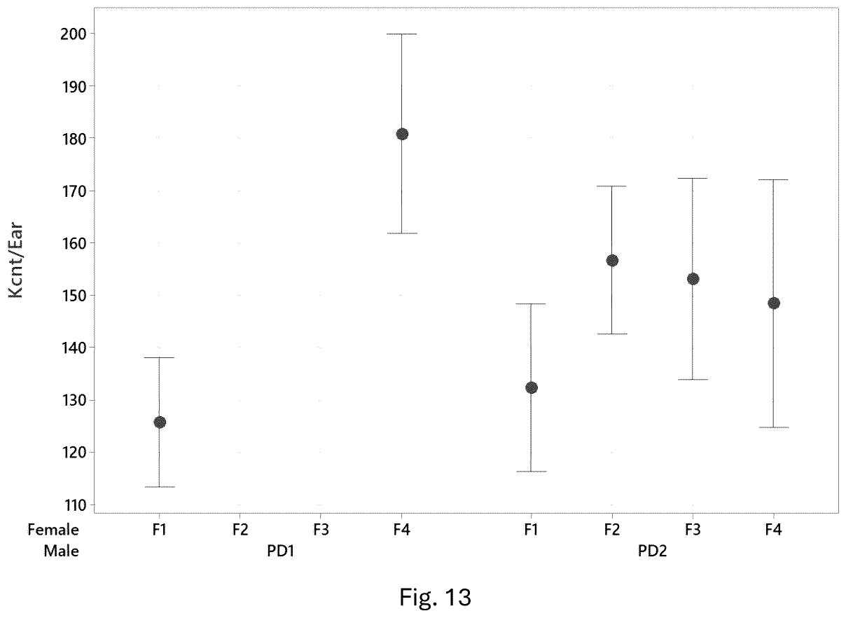

In another embodiment, the invention can be used to measure the current receptivity of female parent to pollen, or any change in receptivity over time. This information can be used to assign a rating or a level of desirability for a given female in a given breeding program or breeding strategy, or to expose limitations of a specific cross between inbreds being evaluated for hybrid seed production. This embodiment is further described in working Example 16. The capacity to quantify the number of pollen grains captured by receptive stigmatic surfaces has obvious benefits for improving the profitability and sustainability of agricultural systems dependent on natural pollination. And it provides a rigorous, timely, and field-based assessment of the need for an intentional pollen application. It also provides novel opportunities to expose genetic variation in efficiency of pollen capture (see FIGS. 12 and 13 in Example 16) which could be related to stigma morphology, stigmatic display, or floral maturation. Such novel information will have direct impact on germplasm selection to advance hybrid seed production. Importantly, pollen on stigma data provide a novel tool to expose the impact of environmental effects on the most critical aspects of reproductive process itself-successful pollen/stigma interactions. When the invention is used to measure pollen grain capture capacity without regard to whether the pollinations are successfully completed, the pollen counts can be conducted almost immediately following natural and/or intentional application of pollen. This is because it is not necessary to wait for the pollen to germinate and begin growing a pollen tube. As a result, it is not necessary to wait 15 minutes or more before removing the pollinated stigmatic tissues for counting the adhered pollen grains. One of skill in the art will be able to determine the necessary wait time following pollination and before sampling the stigmas.

Accordingly, this invention provides the ability to be both prescriptive and predictive. It is predictive because it allows the grower to predict the seed or fruit set anticipated from a given crop. It is also prescriptive because it allows the grower to know the efficiency required to ensure that the seed or fruit set is maximized by providing a “prescription” for the amount of pollen to be applied to the field, whether that is to the entire field, specific areas of the field, or specific rows of the field. This can be based upon various counts conducted in different parts and in different rows of the field. In this way, the invention exceeds the current conventional growing system and its ability to provide the grower with useful information.

The aforementioned analyses and evaluations are all demonstrations of the potential usefulness of the invention in a wide range of circumstances. In all cases, the practice of the invention allows for a much faster result than conventional testing of the same practices, because there is no need to wait for the plants to mature and set seed or fruit. As such, the invention enables a fast and effective means for evaluating treatments, pollinations, equipment, and methods associated with both natural and intentional pollinations.

The invention can be practiced at any time during the period of receptivity of the stigmatic surfaces, or during a period when stigmatic surfaces are exposed but not yet receptive. It can be practiced from the time prior to pollen shed commencing up to and including a time when pollen shed is complete. The timing depends on the intended use of the information and the analyses being conducted after determining the pollination efficiency or contamination level.

As will be appreciated by one of skill in the art, the practice of the invention disclosed herein will provide different benefits depending upon the nature of the crop. For example, some crops have high rates of self-pollination due to pollen being released within the flower even prior to the flower opening. Such crops naturally experience very high rates of self-pollinated seed. Practice of the invention can determine the effectiveness of the self-pollination and help predict seed or fruit set at a time when additional intentional pollinations can still be successfully completed if required to support seed or fruit set.

Although the present invention has been described with reference to the embodiments outlined above, various alternatives, modifications, variations, improvements and/or substantial equivalents, whether known or that are or may be presently foreseen, may become apparent to those having at least ordinary skill in the art. In some instances, in methodologies directly or indirectly set forth herein, various steps and operations are described in one possible order of operation, but those skilled in the art will recognize that steps and operations may be rearranged, replaced, or eliminated without necessarily departing from the spirit and scope of the present invention. Accordingly, the embodiments of the invention set forth above and in the accompanying drawings are intended to be illustrative, not limiting. Persons skilled in the art will recognize that changes may be made in form and detail without departing from the spirit and scope of the invention. Therefore, the invention is intended to embrace all known or earlier developed alternatives, modifications, variations, improvements, and/or substantial equivalents.

The following working examples illustrate the present invention in more detail and are illustrative of how the invention described herein has been demonstrated across a wide range of monocotyledonous and dicotyledonous plant species. The basic method could apply to any crop with crop specific modifications as appropriate.

### Example 1: Counting Pollen Grains from Small Maize Samples without Regard to Saving the Ears

The following procedure has been used routinely to count pollen grains on exposed stigmatic structures (also known as silks) of individual maize ears, 1-3 days after silks first appear. It can be used equally well on a wide variety of plant species.

Within 1 hour after pollination, the tip of a corn ear with pollinated silks was removed from the plant. The plant material was transported to the lab in sealed vials. An aliquot of 40 mL of Isoton™ II diluent solution (Beckman Coulter) (hereafter referred to as Isoton) was placed in a clean 100 ml beaker. The silks, still attached to the ear, were submersed into the Isoton solution and agitated for 60 seconds making sure not to spill any liquid. Subsequently, the washed ear was discarded, and pollen was filtered from the solution using a 50-micron filter. The Isoton solution with pollen grains was poured into a second beaker through a 50-micron filter to capture maize pollen and larger debris). The filtered Isoton solution was poured back into the first beaker to rinse remaining pollen from the beaker and re-filtered. The filter retaining the pollen was then turned over onto a Falcon tube containing 3 mL of Isoton causing the pollen grains to fall into the tube. The end of the filter was covered to prevent liquid from escaping. The Falcon tube was slowly and gently shaken to dislodge the pollen grains from the filter. All the contents of the Falcon tube containing the pollen were then passed into a second Falcon tube through a 100-micron filter, which allowed maize pollen grains to pass but retain larger debris. This tube contained the pollen grains to be measured in a 3 mL volume. The pollen grains were counted using the ‘count’ protocol in an Amphasys impedance flow cytometer according to manufacturer's recommendations.

### Example 2: Counting Pollen Grains from Large Maize Samples with the Intention of Maintaining the Ears on the Plant

The following procedure has been routinely used for large silk plus ear samples (i.e., from individual ears or multiple ears, 3+days after silks first appear) during the research on this invention.

Within 1 hour of pollination, whole silk bouquets from the ear were removed from the plant and placed in capped wide-mouth flasks containing 100 mL to 300 mL of Isoton. All silk tissues were submersed in the Isoton solution and transported to the lab. The flasks were stored up to 24 hours prior to pollen counting. The flasks were agitated with a swirling motion to dislodge pollen from silks, and the liquid was decanted into a second sealable flask. A second, similar volume of Isoton was again added to the silk samples and the agitation was repeated. The washes were combined in the second flask.

The pollen grains per milliliter of liquid were determined using three replicates of 4 mL samples from the solution, which was thoroughly mixed prior to sampling to ensure the pollen was uniformly distributed in the solution. The pollen grains were counted using the ‘count’ protocol in an Amphasys impedance flow cytometer according to manufacturer's recommendations. From this number, the pollen grains per ear could be calculated based on the total sample volume and number of ears sampled.

The following observations were made in the process of repeating the procedures outlined in Examples 1 and 2:
Silk samples were collected up to 48 hours after pollination without a significant loss of pollen counts per silk, although subsequent experiments have shown that silk samples can be collected at any point after pollination but prior to silk senescence.
Maize pollen can remain intact for counting in Isoton solution for long periods prior after collection. The method described was tested up to 24 hours after washing the pollen off the silks.
The method of pollen counting will impact the solution for handling pollen. Isoton solution is compatible with impedance flow cytometry only when used in count mode.
Pollen grains per silk can be calculated from silk counts, if desired.
Pollen grains per kernel can be calculated from the same ears, if pollen is counted at least 24 hours after pollination and pollen/ear is linked to kernel/ear data collected at least 14 days after pollination.

### Example 3: Efficiency of Pollen Removal from Pollinated Maize Stigmas

Silks were collected from 5 ears of maize according to the procedure outlined in Example 2 within 30 minutes of an intentional pollen application to the plants. Samples were processed within 12 hours of sampling. The wash solution was removed from the flask after the first rinse and replaced with 50 mL of Isoton. The washing procedure was repeated 3 times with less than 3% of the total pollen collected in the fourth 50-mL wash. No pollen grains were observed on silks after the fourth wash, suggesting any remaining pollen grains were not adequately rinsed from the washing flask. The 1200 pollen grains collected after the fourth wash was far less than the variation among plant samples (SE=3460 grains). Pollen dosages in this field experiment ranged from 12,900 to 60,400 grains of pollen applied per 5 pooled silk bouquets across 16 randomly selected field samples with a mean+/− standard error of 27898+/−3460 pollen grains per sample.

TABLE 1
Pooled Results from Maize Silks Collected from Five Ears

| | Amphasys | | | % of total | Percentage of |
|---|---|---|---|---|---|
| | pollen | Sample | Pollen | pollen | Total Pollen |
| Rinse | counts | volume | grains | grains | Collected After |
| # | (grains/mL) | (mL) | collected | collected | Each Rinse |
| 1 | 256 | 150 | 38400 | 82.5 | 82.5 |
| 2 | 84 | 50 | 4200 | 9.0 | 91.5 |
| 3 | 55 | 50 | 2750 | 5.9 | 97.4 |
| 4 | 24 | 50 | 1200 | 2.6 | 100.0 |
| | | Total | 46550 | | |

### Example 4: Pollen Removal Relative to Time of Pollination

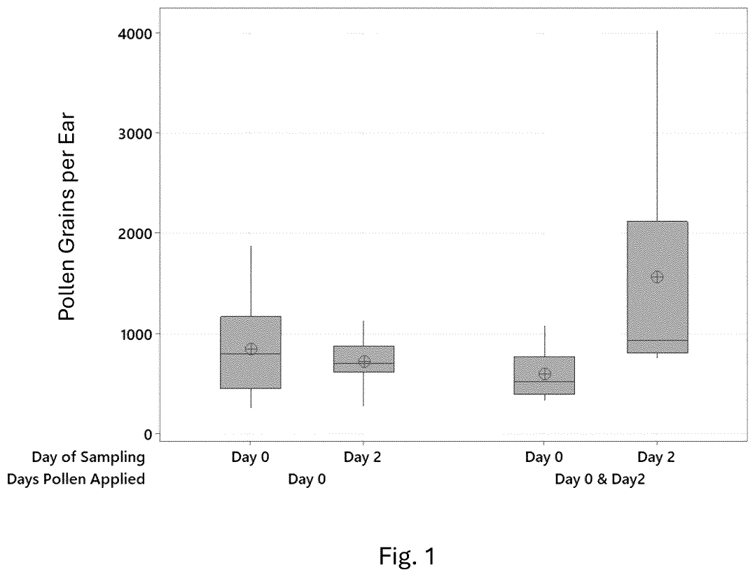

The method outlined in the following example demonstrates that a consistent measure of pollen counts applied to stigmas can be obtained up to 2 days after pollination. FIG. 1 compares counts of pollen grains obtained from bouquets of maize silks pollinated with the same dose of pollen on Day 0 (left) or on Day 0 and again on Day 2 (right).

The ears pollinated only on Day 0 (right) were sampled for pollen counts within 1 hour (Day 0) or after 48 hours (Day 2) after pollination. The ears pollinated on two occasions (Day 0 and Day 2) were sampled within 1 hour of pollination. As expected, the silk bouquets pollinated on one occasion (Day 0 application, Day 0 sampling) contained a similar amount of pollen (about 600 to 800 pollen grains, on average). Surprisingly, the pollen count from silk bouquets sampled 24 hours after pollination was not degraded relative to the ears sampled within 1 hour after pollination.

Likewise, the bouquets pollinated on two occasions (Pollinated Day 0 and Day 2, sampled Day 2) contained about twice as much pollen as those pollinated only once, confirming the pollen counts from the Day 0 application were not degraded. This example demonstrates that it is possible to harvest pollen from ears after fertilization was complete, enabling a direct assessment of pollination efficiency (pollen grains per kernel) as described in Example 5.

### Example 5: Pollen Detection at Extreme Pollination Doses

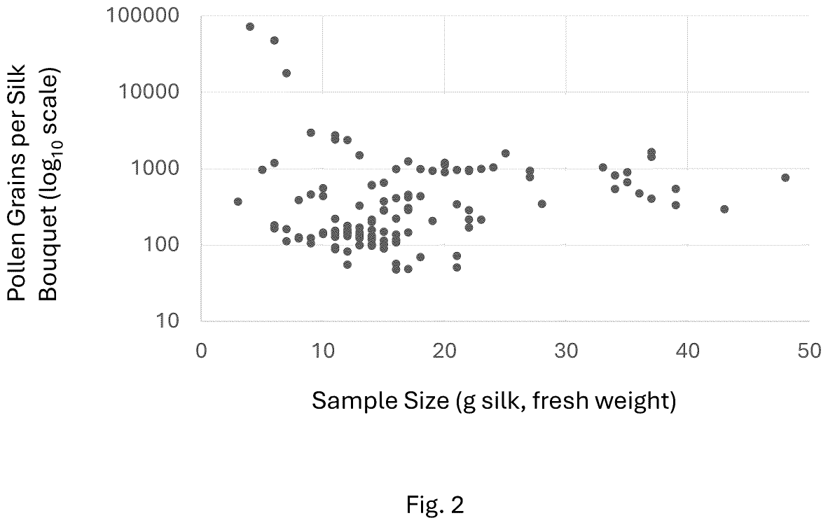

The following example demonstrates that pollen grains can be quantified across a wide range of silk sample sizes and pollen dosages. FIG. 2 presents pollen counts taken from 122 individual or bulked silk bouquets collected from multiple field experiments in 2021. The silk samples varied in size from about 3 to 48 grams fresh weight, and pollen dosages ranged from about 50 to nearly 100,000 grains per silk bouquet. Pollen dosage was varied intentionally using manual applications as well as naturally under field conditions. Bouquet size varied with silk development prior to pollination.

This example demonstrates that pollination efficiency (pollen per silk) can be assessed across a range of pollen dosages and silk bouquet masses that extend well beyond those typically encountered in hybrid seed production or commercial grain production. Furthermore, it is worth noting that ranges of silk bouquet sample sizes and pollen dosages evaluated using this method are much greater than those associated with field-based methods such as described by Lizaso et al. Crop Sci. 43:892-903 (2003) or Westgate et al Crop Sci. 43:934-942 (2003). Accordingly, this method is robust and can be used on a field scale.

### Example 6: Pollen Detection on Wheat Stigmas

An experiment was conducted to compare pollen counts on wheat stigmas. This experiment was conducted on male-sterile wheat plants in a commercial hybrid seed production field near Lakota, ND in July 2022. There were three different treatments: pollen applied in a liquid formulation, pollen applied in a dry formulation containing an additive, or pure pollen applications. Pollen was collected manually between 09:00 and 10:00 from 10 fertile spikes having anthers emerging from the central spikelets (Zadocks stage 65). The collected pollen was filtered through a 50-micron filter to remove anthers and plant debris. Pure pollen then was divided into three application treatments: (1) liquid media, which in this example were designated LQ A, B, or C, with each formulation differing in chemical composition favorable for pollen delivery to stigmas, (2) dry media prepared in accordance with U.S. patent application Ser. No. 16/028,626 to serve as a positive control, and (3) pure pollen which also served as a positive control.

Pure pollen and pollen mixed with 4 parts dry media were applied to exposed stigmas with a small brush; pollen applied in liquid media was transferred to the stigmas in 42 microliters of pollen suspension. For each treatment, pollen was applied to 12 florets on central spikelets of 6 male sterile wheat spikes. Thirty minutes after pollination, stigmas were excised from each floret and pooled into a single sample per spike for pollen counting. Pollen grains were washed from the stigmas and filtered into Amphasys buffer and counted by impedance flow cytometry.

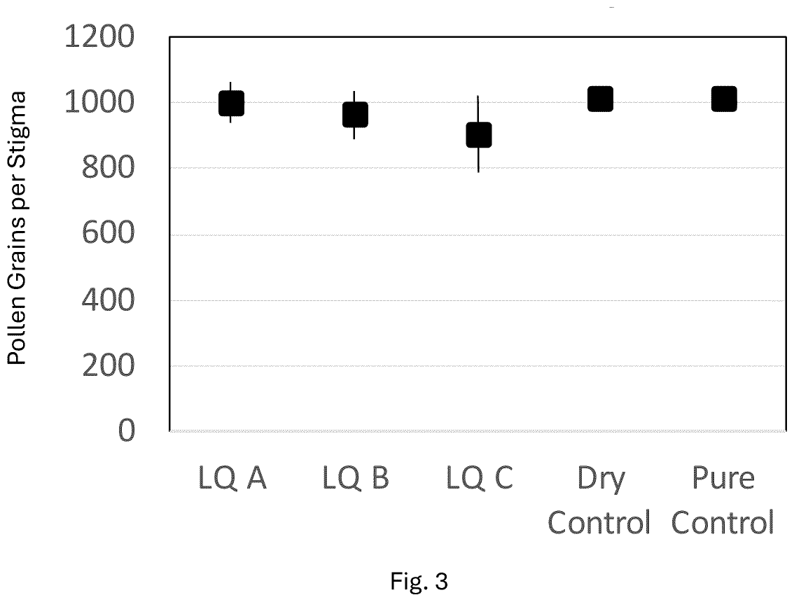

FIG. 3 shows the mean pollen grain count±standard deviation for 6 wheat spikes. These data show the wheat stigmas retained a similar number of pollen grains (about 900 to 1000 grains per stigma) regardless of whether pollen was delivered in a liquid medium, dry medium, or as pure pollen. This unexpected result indicates intentional application of wheat pollen in a liquid or dry format can mimic natural pollination in terms of pollen delivery and retention, the primary determinants of successful pollination in hybrid wheat production.

### Example 7: Mechanical Method of Pollen Application: Dosage Response

This 2021 field experiment tested the effect of pollen dosage on maize seed set in a 12 female rows to one male row configuration. Silk samples were collected within 60 minutes after intentional pollen application and processed according to the procedure outlined in Example 2 within 12 hours of sample collection. Kernels per ear data were collected from adjacent plants within the same plots approximately two weeks after the pollination treatments. The application treatments of untreated, 1 L pollen per acre, 2 L pollen per acre and 4 L pollen per acre were replicated three times, with four sets of silk bouquet samples pooled from 5 randomly selected ears in each rep, for a total of 60 ears sampled.

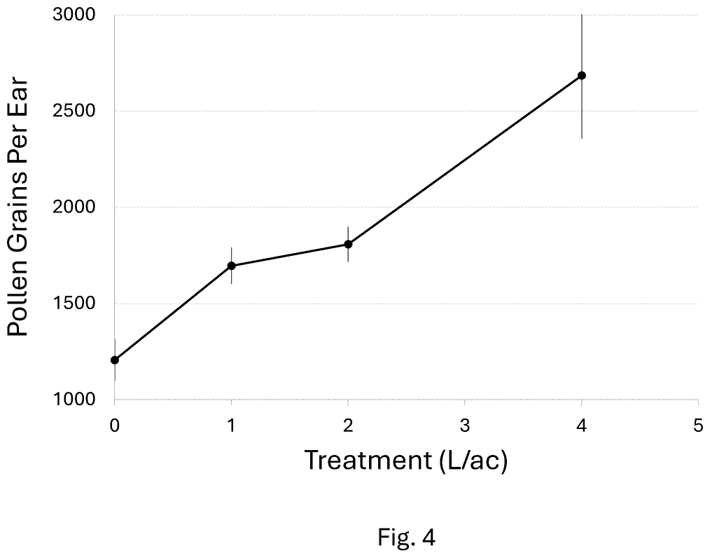

FIG. 4 shows the increase in pollen collected per ear as the application dosage increased. Pollen collected from untreated plants (0 L/ac) reflects the pollen delivered naturally by the male plants in the field. The data are the mean±standard error for three treatment reps having four samples of five ears each.

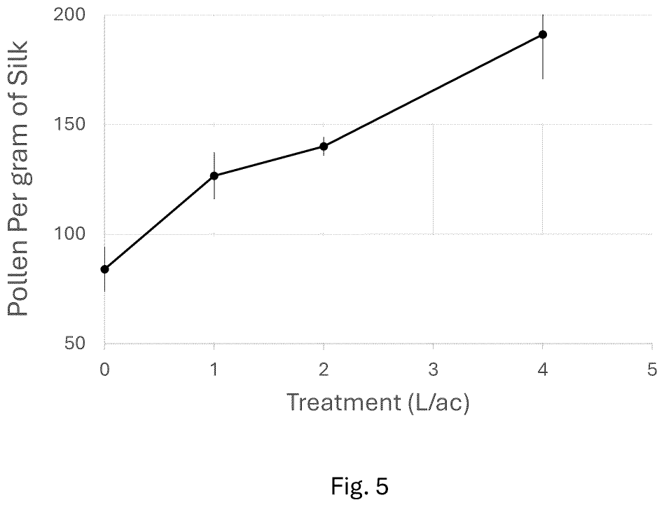

FIG. 5 shows the pollen grains collected per gram fresh weight of silk tissue in each sample. The value normalizes the samples for differences in the mass of the silk bouquets collected from different plants. The data are the mean±standard error of three treatment reps having four samples from 5 plants each.

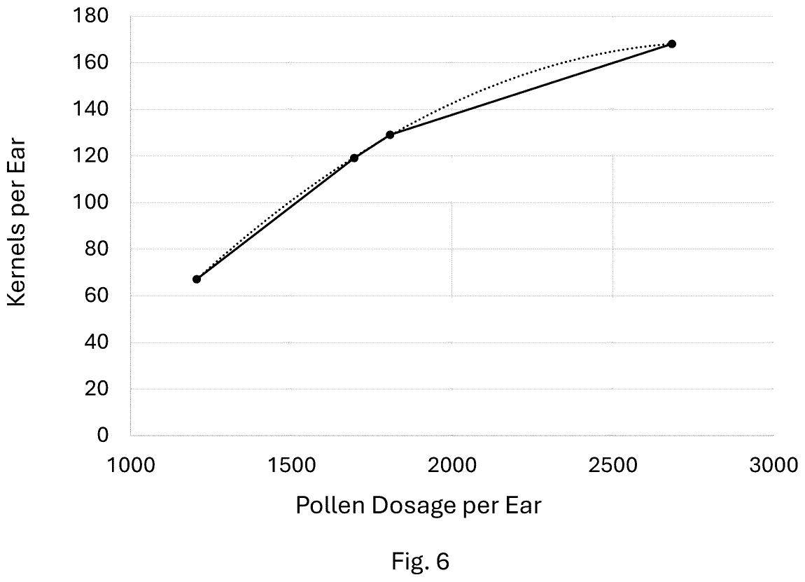

FIG. 6 shows the relationship between pollen dosage per ear and the resulting number of kernels per year (pollination efficiency). These values were calculated from the data presented in FIGS. 4 and 5. The mean number of pollen grains per kernel was 13.7±0.3 across the four treatment doses. The pollination efficiency was nearly identical for the untreated control (14.4 grains/kernel) and the 4 L/ac supplemental pollen treatment (14.0 grains/kernel). This is the first known demonstration of pollination efficiency relating pollen dosage to resulting kernel set following a natural (0 L/ac) and mechanical application of pollen.

### Example 8: Mechanical Method of Pollen Application: Dosage Response

The following data were collected in experiments conducted in Puerto Rico in February 2022. Two experiments were conducted to predict the performance of intentional pollination in terms of kernel formation in response to an increasing dosage of pollen application. Pollen retained by the silk bouquet after application was also measured to quantify the efficacy of mechanical pollination (pollen grains/ear) and efficiency of the applied pollen for kernel formation (pollen grains/kernel) as application dosage increased.

These experiments were conducted in a field arranged as a complete randomized block. The main treatments included pollen applied at 2 L/ac, 4 L/ac, 6 L/ac or 8 L/ac. There were three replications of 10 ears each at each pollen dosage. Pollen was applied beginning at 18:15 hours to ears with two inches of exposed silk length. Doses greater than 2 L/ac were created by sequential passes of the mechanical applicator over the same plants at a dose of 2 L/ac each. For pollen per silk counts, entire ears were harvested from the field within 1 hour of pollination. Pollen was washed off silk bouquets into Isoton solution and counted by impedance flow cytometry as described in Example 1. Kernels per ear were quantified by image analysis on similarly pollinated ears collected from the field at least 14 days after pollination.

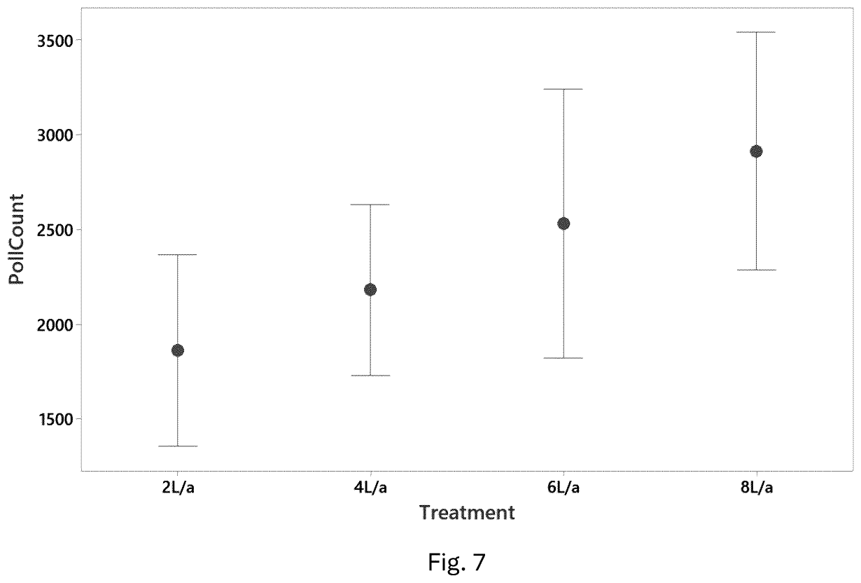

FIG. 7 shows the relationship between the pollen application dosage (in L/ac) and the number of pollen grains collected from the silk bouquets. Data are combined for the two experiments described above and are the mean+95% confidence interval of 6 replicates of 10 ears each. As in the experiments described in Example 4, pollen captured by the silk bouquets increases with application dosage, but these data extend this relationship up to an application rate of 8 L/ac.

### Example 9: Efficiency of Kernel Set with Increasing Pollen Dose

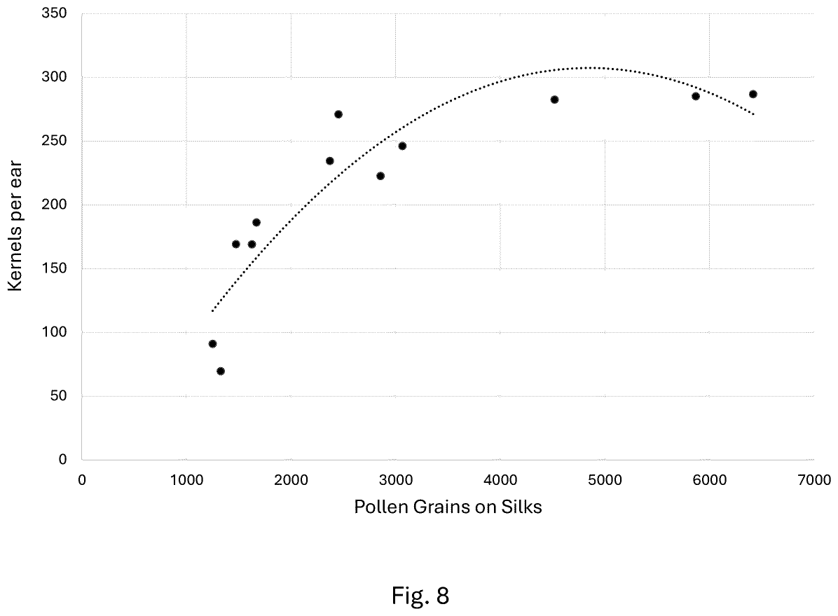

In order to study the efficiency of kernel set in maize with an increasing pollen dose, charts were created using the data generated in previous working examples. FIG. 8 shows the relationship between pollen retained on the silk bouquet of each ear and resulting kernel set on the same ears. Each data point is the mean of 10 ears mechanically pollinated at dosages ranging from 2 to 8 L/ac. The response to pollen dosage is nearly linear up to about 3000 pollen grains/ear, as observed in the experiments described in Examples 7 and 8. The efficiency of kernel set (pollen grains/kernel) decreases with the number of pollen grains captured by the silk bouquet. Efficiency values are estimated from the fitted curve. The equation of the fitted curve is: y=−1E-05x²+0.1425x−38.297, R²=0.8357.

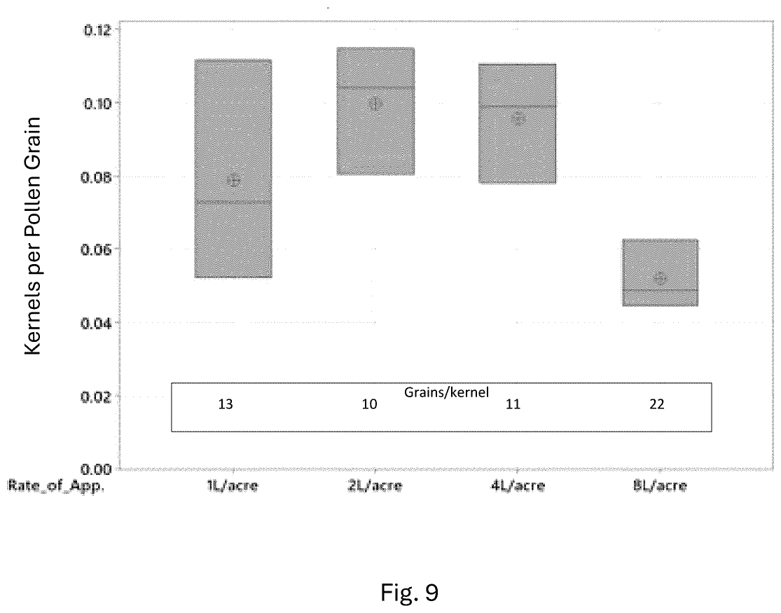

FIG. 9 is a box-plot representation of the efficiency of kernel set pooled for each level of mechanical pollen dosage. The efficiencies, shown as kernels per pollen grain captured on the vertical axis and as pollen grains per kernel in the inset, are greatest at the moderate pollen dosages of 2 and 4 L/ac. Efficiency values are estimated from the mean values of kernels/pollen applied (circles in the box plot) for each application dosage. These experiments together show the maximum efficiency of kernel set with mechanical application of preserved pollen was about 10 to 12 grains per kernel. Efficiency (grains/kernel) decreased dramatically at saturating pollen dosages greater than 3000 grains per silk bouquet.

### Example 10: Predicting Kernel Set from Pollen Counts After Pollination

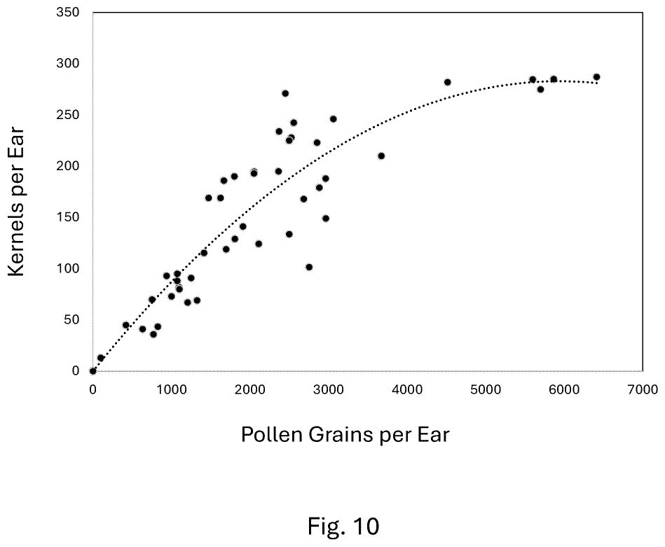

A composite of data collected from field experiments conducted from 2021 to 2023 in Iowa, Texas, and Puerto Rico revealed a fairly consistent and robust relationship between the amount of pollen captured by silk bouquets and the resulting number of kernels produced by maize inbred plants. This relationship, shown in FIG. 10, includes data collected from natural pollination events with fresh pollen as well as from mechanical applications with fresh and preserved pollen. As demonstrated in Examples 7 and 9 above, the relationship between pollen per ear and kernels per ear was fairly linear and most efficient at about 10 to 12 pollen grains/kernel for pollen counts up to about 3000 grains per silk bouquet. Efficiency of kernel set decreased to about 22 pollen grains at pollen doses over 6000 grains per ear. This novel and robust relationship enables one of skill in the art to predict the eventual kernel yield of naturally and/or intentionally pollinated plants immediately after pollination is complete. Typically, yield assessment is relegated to harvest of mature kernels many weeks after pollination. Thus, the described method provides a quantitative mid-season evaluation of productivity not currently available to seed, grain, or fruit producers. This information has a direct impact on forecasting harvesting logistics, product market value, and sales strategy.

### Example 11: Pollen Per Stigma of Three Dicot Species Resulting from Natural and Manual Pollination

These data were collected in February 2024 on flowering plants purchased from a local flower shop and grown in the PowerPollen greenhouse until sampled. Chrysanthemum (Chrysanthemum indicum), Kalanchoe (Kalanchoe blossfeldiana), and African Violet (Streptocarpus ionanthus) plants all produce perfect flowers (i.e., contain both staminate (male) and pistillate (female) floral parts). The Chrysanthemum rachis produces both disc (central) and ray (peripheral) flowers. These flower types were analyzed separately. Stigmas collected from Chrysanthemum and Kalanchoe flowers were self-pollinated; thus pollen per stigma values reflect natural pollen values. African Violet flowers were intentionally pollinated (manually) by gently brushing pollen-bearing stamens across exposed pistils to transfer pollen to the stigmas. Pollinated stigmas were pooled from a number of flowers ranging from 20 to 160, depending on species. Two, four, and one stigma per flower were sampled on Chrysanthemum, Kalanchoe, and African Violet flowers, respectively, reflecting the anatomical variation among species.

Harvested stigmas were transferred to 1 mL of Isoton in a 4-mL microfuge tube and stored at 4° C. until analyzed. The pollen extraction and filtering process followed the procedures outlined for recalcitrant pollen in Example 1, except a 50-micron filter was employed to isolate Kalanchoe and African Violet pollen and a 100 micron filter was used for Chrysanthemum pollen. Total pollen counts per pooled sample were quantified by impedance flow cytometry.

Table 2 shows self-pollinated stigmas of both ray and disc Chrysanthemum flowers contained 27 to 30 pollen grains per stigma. Self-pollinated Kalanchoe flowers contained 9 to 14 pollen grains per stigma. Manually pollinated stigmas of African Violet flowers were intermediate, with 23 pollen grains per stigma. These data indicate the pollen collection and counting process described herein can be used successfully to assess the pollination intensity on dicotyledonous flowers, whether pollinated naturally or intentionally.

TABLE 2
Pollen counts on stigmas of self-pollinated Chrysanthemum and Kalanchoe
flowers and intentionally-pollinated African Violet flowers.

| | | | | | | | Pollen |
|---|---|---|---|---|---|---|---|
| | Pollen | Pollen | Stigma | Method of | Sigmas | Pollen | per |
| Dicot species | Type | Source | Type | Pollination | pooled | counts | stigma |
| Chrysanthemum | orthodox | 55 Disc | Semi- | Natural | 110 | 3192 | 29 |
| | | flowers | dry | | | | |
| Chrysanthemum | orthodox | 50 Disc | Semi- | Natural | 100 | 3048 | 30 |
| | | flowers | dry | | | | |
| Chrysanthemum | orthodox | 55 Ray | Semi- | Natural | 110 | 3003 | 27 |
| | | flowers | dry | | | | |
| Kalanchoe | orthodox | 10 open | Semi- | Natural | 40 | 342 | 9 |
| | | flowers | dry | | | | |
| Kalanchoe | orthodox | 40 open | Semi- | Natural | 160 | 2271 | 14 |
| | | flowers | dry | | | | |
| African Violet | orthodox | 20 open | Dry | Intentional | 20 | 465 | 23 |
| | | flowers | | | | | |

### Example 12: Pollen Per Stigma and Pollen Per Seed Resulting from Intentional Pollination of Cucurbit Species

These data were collected in March of 2024 on flowering plants grown from seed in the greenhouse. Summer squash (Cucurbita pepo), winter squash (Cucurbita moschata), and cucumber (Cucumis sativus) plants all produce imperfect flowers containing either staminate (male) or pistillate (female) floral parts. In all cases, female flowers with receptive stigmas were intentionally pollinated (manually) by gently brushing pollen-bearing stamens collected from male flowers across the exposed stigmas of receptive female flowers. Pollinated stigmas were pooled for pollen counts from three flowers. One to three stamens were pooled for pollinations. Stigmas were sampled 24 to 48 hours after pollination for pollen counts; fruits were harvested approximately 21 days after pollination for seed counts.

Harvested stigmas were transferred to 1 mL of Isoton in a 4-mL microfuge tube and stored at 4° C. until analyzed. The pollen extraction and filtering process followed the procedures outlined for recalcitrant pollen in Example 1, except a 50-micron filter was employed to isolate cucumber pollen; a 100 micron filter was used for summer and winter squash pollen. Total pollen counts per pooled sample were quantified by impedance flow cytometry.

Table 3 shows intentional pollinations transferred approximately 9000 to 12000 pollen grains to summer squash stigmas, approximately 3700 to 5300 pollen grains to winter squash stigmas, and approximately 1300 to 4300 pollen grains to cucumber stigmas. All pollinations successfully produced fruit bearing seed numbers typical of the species; approximately 200 to 300 seed per fruit for summer squash, approximately 50 to 80 seed per fruit for winter squash, and approximately 110 to 190 seed per fruit for cucumber. These data indicate the pollen collection and counting process described herein can be used successfully to assess both the intensity of pollination (pollen per stigma) and the efficiency of pollen (pollen per seed) in seed production of dicotyledonous Cucurbitaceae plants. Since manual pollinations were made by brushing floral style tissues (pollen bearing) across stigmatic surfaces of female only or emasculated flowers, the variation in pollen counts per stigma across these species may reflect inherent differences in pollen load per anther, pollen self-adhesion characteristics, stigma morphology, or kinetics of physical pollen transfer. It was not the intent of this analysis to resolve these morphological and physiological differences, but the invention can be used to provide data required to resolve those differences or gain greater understanding of genotype-specific pollen capture efficiency.

TABLE 3
Pollen counts on stigmas and pollen grains per seed produced by intentionally
pollinated flowers of summer squash (Cucurbita pepo), winter squash
(Cucurbita moschata), and cucumber (Cucumis sativus) plants.

| | | | Pollen | | | | | | |
|---|---|---|---|---|---|---|---|---|---|
| | | | Source | Method of | | | Pollen | Seeds | Pollen |
| Dicot | Pollen | Stigma | # male | Intentional | Stigmas | Pollen | per | Produced | per |
| species | Type | Type | flowers | Pollination | pooled | count | stigma | per fruit | seed |
| Summer | recalcitrant | Semi- | 3 | Manual | 3 | 27570 | 9190 | 200 | 46 |
| squash | | dry | | | | | | | |
| Summer | recalcitrant | Semi- | 3 | Manual | 3 | 35580 | 11860 | 290 | 41 |
| squash | | dry | | | | | | | |
| Summer | recalcitrant | Semi- | 3 | Manual | 3 | 29160 | 9720 | 240 | 41 |
| squash | | dry | | | | | | | |
| Winter | recalcitrant | Semi- | 3 | Manual | 3 | 11040 | 3680 | 49 | 75 |
| Squash | | dry | | | | | | | |
| Winter | recalcitrant | Semi- | 3 | Manual | 3 | 16050 | 5350 | 80 | 67 |
| Squash | | dry | | | | | | | |
| Cucumber | recalcitrant | Semi- | 1 | Manual | 1 | 1324 | 1324 | 115 | 12 |
| | | dry | | | | | | | |
| Cucumber | recalcitrant | Semi- | 2 | Manual | 1 | 4350 | 4350 | 132 | 33 |
| | | dry | | | | | | | |
| Cucumber | recalcitrant | Semi- | 3 | Manual | 1 | 3510 | 3510 | 189 | 19 |
| | | dry | | | | | | | |

### Example 13: Pollen Per Stigma Collected from Naturally Pollinated and Intentionally Pollinated Hybrid Petunia (Petunia×Hybrida) Flowers

These data were collected in April of 2024 on flowering petunia (Petunia×hybrida) plants grown from seed in a greenhouse located in Ames, IA. Petunia produces perfect flowers containing both staminate (male) or pistillate (female) floral parts. Therefore, some flowers were emasculated so that intentional pollinations could be conducted, while others were left intact to permit natural pollinations. Pollen counts on receptive stigmas were quantified both for naturally self-pollinated flowers and emasculated flowers pollinated intentionally (manually). Intentionally pollinated plants were emasculated between 10:00 am and 4:00 pm on the day prior to pollination. These flowers were then intentionally pollinated between 9 and 10 am the following morning by gently brushing pollen-bearing stamens collected immediately prior to pollination across the stigmas. Stigmas were pooled from two to six flowers for pollen counts. Two to six stamens were pooled for intentional pollinations (typically from one flower). Stigmas were sampled from 1 to 30 hours after pollination for pollen counts.

Harvested stigmas were transferred to 1 mL of Isoton in a 4-mL microfuge tube and stored at 4° C. until analyzed. The pollen extraction and filtering process followed the procedures outlined for recalcitrant pollen in Example 1, except a 20-micron filter was employed to isolate petunia pollen. Total pollen counts per pooled sample were quantified by impedance flow cytometry.

Table 4 shows the naturally self-pollinated stigmas contained approximately 70 pollen grains per stigma, on average, consistent with published values (Santos de Souza, et al, (2022) Acta Botanica Brasilica, Vol 35, <doi: 10.1590/0102-33062021abb0209>). Intentional pollination transferred approximately 250 pollen grains to the stigmas, on average. Evidently, pollen counts per stigma were stable at least up to 48 hours after pollination. These data further confirm the pollen collection and counting process described herein can be used successfully to assess intensity of pollination (pollen per stigma) of dicotyledonous Petunia plants, whether assessed on naturally self-pollinated or intentionally pollinated flowers.

TABLE 4
Pollen counts on stigmas of naturally pollinated and intentionally
pollinated flowers of hybrid petunia (Petunia × hybrida).

| | | | | | | | | Hours |
|---|---|---|---|---|---|---|---|---|
| | | | | | | | | after |
| | | | Pollen | | | | Pollen | pollination |
| Dicot | Pollen | Stigma | Source | Method of | Stigmas | Pollen | per | stigmas |
| species | Type | Type | # anthers | Pollination | pooled | count | stigma | sampled |
| Petunia | orthodox | wet | 3 | Natural | 3 | 199 | 66 | 48 |
| Petunia | orthodox | wet | 2 | Natural | 2 | 164 | 82 | 48 |
| Petunia | orthodox | wet | 2 | Natural | 2 | 122 | 61 | 24 |
| Petunia | orthodox | wet | 5 | Intentional | 5 | 1152 | 230 | 6 |
| Petunia | orthodox | wet | 6 | Intentional | 6 | 1244 | 207 | 24 |
| Petunia | orthodox | wet | 4 | Intentional | 4 | 1578 | 395 | 30 |
| Petunia | orthodox | wet | 3 | Intentional | 3 | 524 | 181 | 6 |

### Example 14: Seed Set and Estimated Pollen Per Seed Resulting from Intentional Pollination of Hybrid Petunia (Petunia×Hybrida) Flowers

These data were collected in April of 2024 on flowering petunia (Petunia×hybrida) plants grown from seed in the greenhouse. As in the previous example, the plants were emasculated between 10:00 am and 4:00 pm on the day prior to intentional pollination. In most cases, flowers were intentionally pollinated between 9 and 10 am the following morning by gently brushing pollen-bearing stamens collected immediately prior to pollination across the stigmas. Two to six stamens were pooled for intentional pollinations (typically collected from one flower). In five cases, stigmas were removed 24 to 48 hours after pollination to determine when stigmas also could be harvested for pollen counts without disrupting seed set. Seed pods were harvested approximately 21 days after pollination for seed counts.

Table 5 shows all combinations of pollinations between genetically distinct petunia plants produced abundant seed with an average of approximately 230 seeds per pod (N=16, STDEV=56.3)−values typical for petunia (Santos de Souza, et al, (2022) Acta Botanica Brasilica, Vol 35, <doi: 10.1590/0102-33062021abb0209>). As noted, this calculation assumes emasculated stigmas received a similar number of pollen grains as quantified in the previous example (253 grains per stigma on average). Surprisingly, emasculated flowers were very efficient at forming seeds as only 1.2 pollen grains per seed were required, on average (N=16, STDEV=0.4). It also is noteworthy that the late afternoon intentional pollinations on 01/04 and 06/04 were equally successful, as was the intentional pollination made on 11/04, more than 40 hours after emasculation. A novel discovery was that removing stigmas 24 to 48 hours after pollinations had no impact on seed formation, which confirms that the described invention now makes it possible to assess both the intensity of pollination (pollen per stigma) and the efficiency of pollen (pollen per seed) in seed production of dicotyledonous Petunia plants.

TABLE 5
Seed set and estimated pollen per seed resulting from intentional
pollination of hybrid petunia (Petunia × hybrida) flowers.

| | | | | | | | Hours |
|---|---|---|---|---|---|---|---|
| | | | | | | Estimated | after |
| | | | | | Seeds | pollen | pollination |
| Dicot | Female | Male | Date/time of | Date/time of | per | grains | stigma |
| species | flower | flower | Emasculation | Pollination | Pod | per seed* | removed |
| Petunia | Purple | White | 1 Apr. 2024 | 2 Apr. 2024 | 300 | 0.8 | |
| | | | 15:30 | 15:00 | | | |
| Petunia | Light | Dark | 3 Apr. 2024 | 4 Apr. 2024 | 210 | 1.2 | 24 |
| | Purple | pink | 11:30 | 10:00 | | | |
| Petunia | White | Dark | 3 Apr. 2024 | 4 Apr. 2024 | 315 | 0.8 | 48 |
| | | pink | 11:30 | 10:00 | | | |
| Petunia | Purple | White | 3 Apr. 2024 | 4 Apr. 2024 | 250 | 1.0 | 24 |
| | | | 11:30 | 10:00 | | | |
| Petunia | Purple | Light | 4 Apr. 2024 | 5 May 2024 | 300 | 0.8 | |
| | | pink | 10:30 | 09:30 | | | |
| Petunia | White | Purple | 4 Apr. 2024 | 5 May 2024 | 200 | 1.3 | |
| | | | 10:30 | 09:15 | | | |
| Petunia | Purple | Light | 5 Apr. 2024 | 6 Apr. 2024 | 115 | 2.2 | |
| | | pink | 12:00 | 15:30 | | | |
| Petunia | Purple | White | 8 Apr. 2024 | 9 Apr. 2024 | 165 | 1.5 | 30 |
| | | | 12:30 | 09:30 | | | |
| Petunia | Purple | Light | 8 Apr. 2024 | 9 Apr. 2024 | 275 | 0.9 | 30 |
| | | pink | 12:50 | 09:30 | | | |
| Petunia | Light | White | 9 Apr. 2024 | 11 Apr. 2024 | 215 | 1.2 | |
| | purple | | 16:00 | 09:00 | | | |
| Petunia | White | Purple | 9 Apr. 2024 | 10 Apr. 2024 | 220 | 1.2 | |
| | | | 16:00 | 09:00 | | | |
| Petunia | Pink | White | 10 Apr. 2024 | 11 Apr. 2024 | 210 | 1.2 | |
| | | | 16:00 | 09:00 | | | |
| Petunia | Pink | Purple | 15 Apr. 2024 | 16 Apr. 2024 | 220 | 1.2 | |
| | | | 14:00 | 09:00 | | | |
| Petunia | Purple | Dark | 15 Apr. 2024 | 16 Apr. 2024 | 210 | 1.2 | |
| | | pink | 14:00 | 09:00 | | | |
| Petunia | Purple | Dark | 15 Apr. 2024 | 16 Apr. 2024 | 305 | 0.8 | |
| | | pink | 14:00 | 09:00 | | | |
| Petunia | Bright | Purple | 15 Apr. 2024 | 16 Apr. 2024 | 180 | 1.4 | |
| | pink | | 14:00 | 09:00 | | | |

*assumes an average of 253 pollen per stigma observed for intentional pollinations in Example 13.

### Example 15: Calculating Pollination Efficiency

The following example outlines how to develop a predictive model for determining estimated seed set in any given crop, although this example uses corn for the crop type. Any other crop estimated seed or fruit set can be calculated using the same technique. Studies were conducted to capture and measure total grains of pollen on a given stigmatic surface. In addition, the studies then measured the actual seed yield generated by each stigmatic surface. By collecting pollen from stigmatic surfaces containing anywhere from 0 through to more than 15,000 grains of pollen, and then harvesting the same seed or fruit from the plant after development, an equation can be developed that has a high correlation to total seed or fruit set.

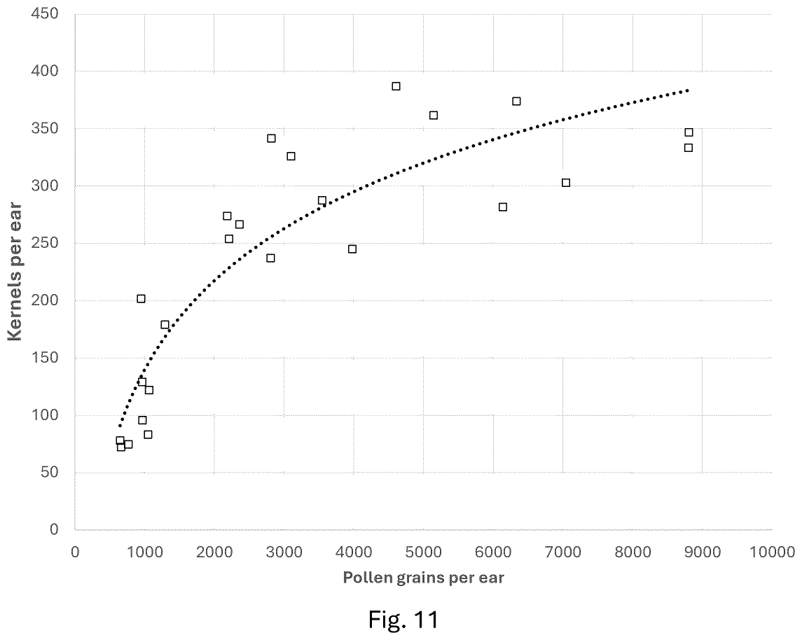

Data were collected from a series of related field experiments conducted in Puerto Rico, Texas, and Iowa in 2022 and 2023 to determine the efficiency with which pollen delivered to exposed and receptive stigmatic surfaces translated to actual seed yield on individual corn ears. Pollen captured on exposed stigma (silk) bouquets was quantified by the methods described in Examples 1 and 2. Resulting kernels per ear were measured at least 14 days after pollination. Pollen dosages per were varied naturally by selecting open-pollinated female plants throughout the pollen shed period, and varied intentionally via metered manual pollination to provide a wide range of dosages that might impact the efficiency of pollination. As an example specific to corn, FIG. 11 shows the graph associated with the equation developed to predict estimated yield. In this case of corn, which is a compilation of several field experiments, the following logarithmic model was determined:

$$Estimated yield = 109 ln ( x ) - 570$$

To obtain a natural logarithmic model, a linear regression analysis was performed where the dependent variable (kernels per ear) is the natural logarithm of the original dependent variable and the independent variable (pollen grains) remains as is, resulting in an equation of the form: In (y)=a+bx*; where “a” is the intercept and “b” represents the slope of the line. This model was calculated using standard linear regression methods on the data set where the y-values are transformed using the natural logarithm function (In).

The logarithmic model was selected for this work as it tends to change dramatically at low levels of the independent variable and changes slowly at higher levels, adjusting well to some biological functions.

It is evident from the graph that after 5,000 grains of pollen have been reached, the ear of corn is considered to be at full capacity of kernels for ears which can carry around 400 seeds. This particular example was on inbred ears, which tend to carry fewer seeds than a hybrid ear. Similarly, when the number of pollen grains totals slightly less than 1,000, the ear only generates about 75 to 80 kernels.

### Example 16: Genotype Effect on Stigma Pollen Capture and Yield

A pollen capture capacity experiment was conducted in Puerto Rico in March 2022. In this experiment, pollen from two pollen-donor genotypes was applied to 4 different female genotypes using a delivery method that measures the exact volume of pollen applied. A single application of 14 μl of preserved pollen from one of the pollen donor genotypes was applied to the female plants while the female flower was receptive and fertile. A total of 45 plants for each female genotype were pollinated with the metered system. Forty-eight hours following the intentional pollen application, all exposed stigmatic surfaces of all 45 female plants from each genotype for each pollen-donor were removed and sent to the laboratory for pollen counting. The remaining stigmatic surfaces (not exposed) were tagged and labeled to accord with the removed stigmatic material to enable direct coordination of results between pollen counts and effective kernel numbers per ear. Pollen was from removed stigmatic surfaces and quantified using Amphasys as described in Example 1 and in other examples. The kernel numbers (see FIG. 13) were obtained one month after pollination. Because each plant was labeled after stigma removal, a direct connection between pollen counts and kernel number per ear could be established.

FIG. 12 shows the number of pollen grains captured by the exposed stigmas (silks) varied dramatically across female parent lines for both pollen donor parents (PD1 and PD2). Female parent F4 captured nearly 50% more pollen than the female parent F1, despite plants being carefully selected for uniform stigma (silk) display and the application of a similar volume of pollen per pollination. Female parents F2 and F3 were intermediate in pollen capture capacity.

FIG. 13 shows the actual kernel counts per ear on the same female target parents shown in FIG. 12, resulting from pollinations by the same two pollen donors. In general, the variation in pollen capture capacity for each combination of pollen donor and female parent was reflected in the kernels per ear, with F8 producing nearly 50% more kernels than F1 when pollinated by male donor PD1. Kernel formation was less than expected, however, for F4 pollinated by PD2, based on the pollen on stigma counts. This outcome exposes a pollen donor×female parent interaction not obvious from reciprocal crosses between inbreds typical of a commercial inbred selection program. Evaluation of pollen counts on stigmatic tissue can be used to rate the pollen capture capacity of female flowers, genotype by genotype interactions for pollination efficiency, as well as predicting yield responses well in advance of kernel maturity.

### Example 17: Demonstration of Pollen Counting Following Natural and Intentional Pollinations

This experiment was conducted in Texas during the month of May 2023. A hybrid seed field was configured with 10 male sterile female rows (target parent plants) planted between two male rows (pollen donor parent plants). The Male-East and Male-West rows served as a pollen donors to the 10 intervening rows of male sterile female corn plants, rows F1-F10. This configuration of male and female rows is an extreme arrangement designed to generate variation in pollen counts per ear across female plant rows.

Pollen counts per ear were measured during the early stages of pollen shed as described in Example 1 and 2. Collected pollen per ear was quantified by Amphasys impedance flow cytometry. The dotted (lower) line shows the average pollen counts per ear for naturally pollinated female plants in each row (Natural PC). Note that pollen counts for Row F1 were more than twice that of the other female rows reflecting the persistent wind coming from the east during pollen shed.

Based on these measured deficits for pollen on stigmas throughout rows F1-F10, an intentional mechanical application of previously collected and stored pollen was made on all rows, including the male rows. Post application, pollen was again counted on stigmatic surfaces (using Amphasys) to measure the total pollen present on stigmas from the combination of natural pollen plus the application with preserved pollen (Nat+Commercial PC). As expected, these pollen counts confirmed the mechanical application of pollen substantially increased the amount of pollen on stigmas in comparison to the amount of pollen adhering to stigmas during natural pollination across all rows.

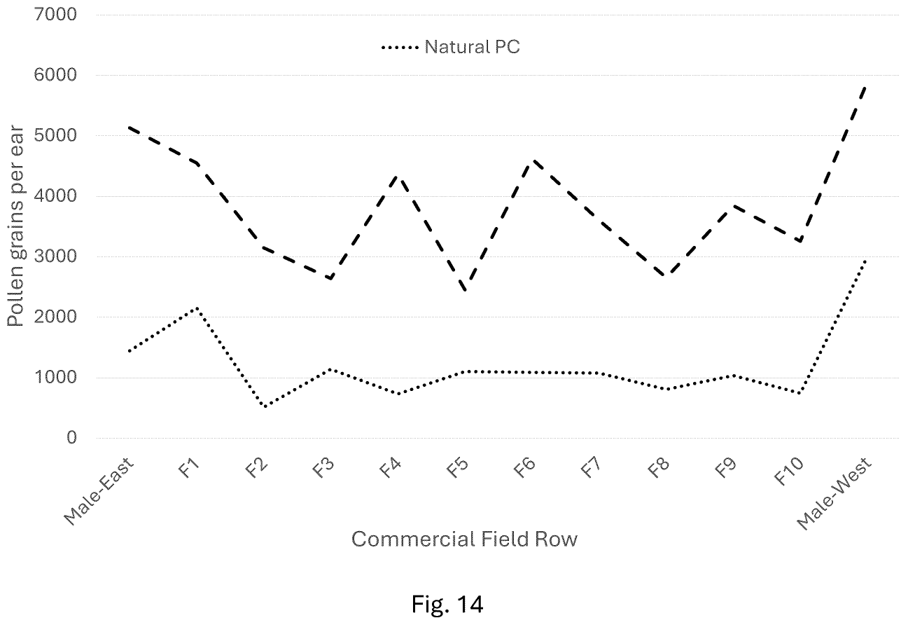

The data derived from this experiment is shown in FIG. 14. The x-axis shows the two male rows as Male-East and Male-West, and the ten female rows as F1 through F10. Male-East and Male-West rows served as a pollen donors to the 10 intervening rows of male-sterile female corn plants, rows F1-F10, which were the target parent plants. The y-axis shows pollen counts per ear. The dotted (lower) line shows the average pollen counts per ear for naturally pollinated female plants in each row (Natural PC). Note that pollen counts for Row F1 were more than twice that of the other female rows reflecting the persistent wind coming from the east during pollen shed. Based on these measured deficits for pollen on stigmas throughout rows F1-F10, an intentional mechanical application of previously collected and stored pollen was made on all rows, including the male rows.

Post application, a second count of pollen on stigmatic tissue was conducted to measure the total pollen present on the exposed stigmas from the combination of natural pollen plus the application with preserved pollen (Nat+Commercial PC). These pollen counts confirmed the mechanical application of pollen increased pollen on stigmas substantially over natural pollination across all rows.

### Example 18: Prediction of Kernels Per Ear at Harvest

This example demonstrates the ability to accurately predict the number of kernels per ear at harvest based on the stigmatic tissue pollen counts acquired following pollination. Predicted values in this experiment were compared to measured kernels per ear at harvest maturity on the same treated plants, maintaining direct identity of ears during both pollen counts and kernel counts. This example was a continuation of the experiment described in Example 17, and thus used the same field with Male-East and Male-West (pollen donor parent) rows serving as a pollen donors to 10 intervening rows of male sterile female corn (target parent plants), rows F1-F10. Fifteen days after the intentional pollination was conducted (as described in Example 17), the ears that had been labeled and used for pollen counts were removed from plants and the kernels were counted.

The estimated values of kernels based on the pollen counts described in Example 17 and the actual number of kernels obtained in this example were compared to determine the accuracy of the predictions.

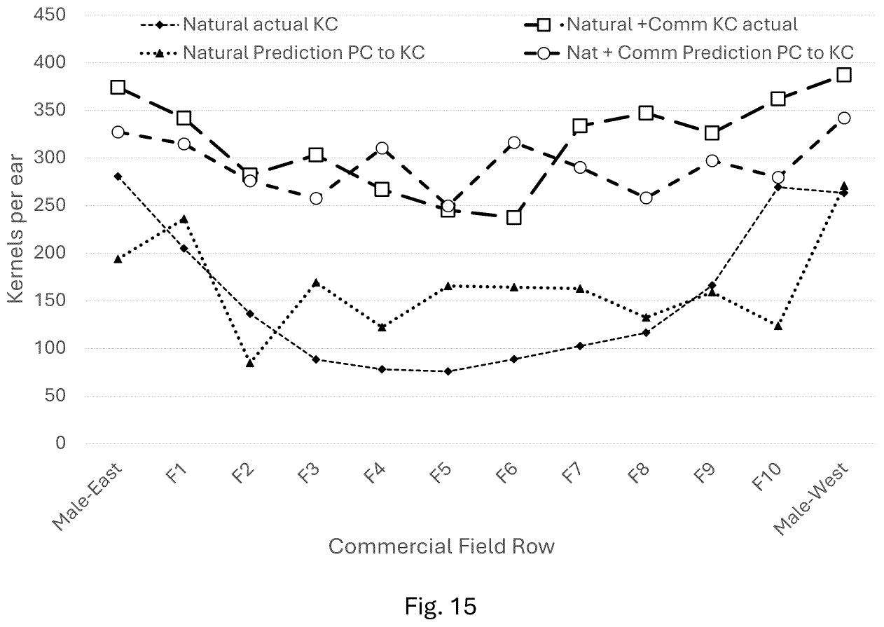

The predicted number of kernels per ear based on pollen counts per stigma was calculated using the predictive model for pollen presence and yield (logarithmic curve) described in Example 15. FIG. 15 presents the data gathered from the experiment. The four curves in FIG. 15 present the kernel counts (KC) per ear measured at harvest for the naturally-pollinated plants (Natural Actual KC), the kernel counts per ear predicted from pollen counts (PC) for naturally-pollinated plants (Natural Prediction PC to KC), the kernel counts per ear measured at harvest for the natural+commercially-pollinated plants (Natural+Commercial KC), and the kernel counts per ear predicted from pollen counts for naturally+commercially-pollinated plants (Natural+Commercial Prediction PC to KC) for each of the male and female rows.

Both the measured and predicted values for kernels per ear for the naturally-pollinated plants (lower set of curves) confirm the pollen deficit in female rows distant from the male rows. Predicted kernel counts per ear were overestimated for the female rows with very low pollen on stigma counts. This may reflect the potential for greater prediction error associated with the steep slope at low pollen count values in the predictive model (FIG. 11), the development of which was described in Example 15. Nonetheless, measured and predicted values were well correlated (r=0.49).

Likewise, both the measured and predicted values for kernels per ear for the natural+commercially pollinated plants (upper set of curves) showed the intentional pollination resulted in a substantial increase in kernels per ear. The correlation between measured and predicted kernels per ear was r=0.32. The measured increase in kernels per ear averaged across all male and female rows was 159±14 (M+SE), while the average predicted increase was 128±12 (M+SE) kernels per ear. Predicted kernel counts tended to underestimate measured kernels per ear in female rows nearest the Male-East and Male-West rows, which may reflect the decreased resolution of the prediction model (FIG. 11) at higher pollen loads. Finally, it is important to note the prediction for supplemental pollen application varied for each female row, which enables a prescriptive approach to predicting and delivering a timely supplemental pollination needed to achieve profitable seed yield. Taken together, the results presented in FIGS. 14 and 15 demonstrate the potential to predict the number of kernels per ear at harvest from the pollen on stigma data acquired during pollination in a commercially-relevant field environment.

### Example 19: Use of Fluorescent Microscope for Pollen Counts

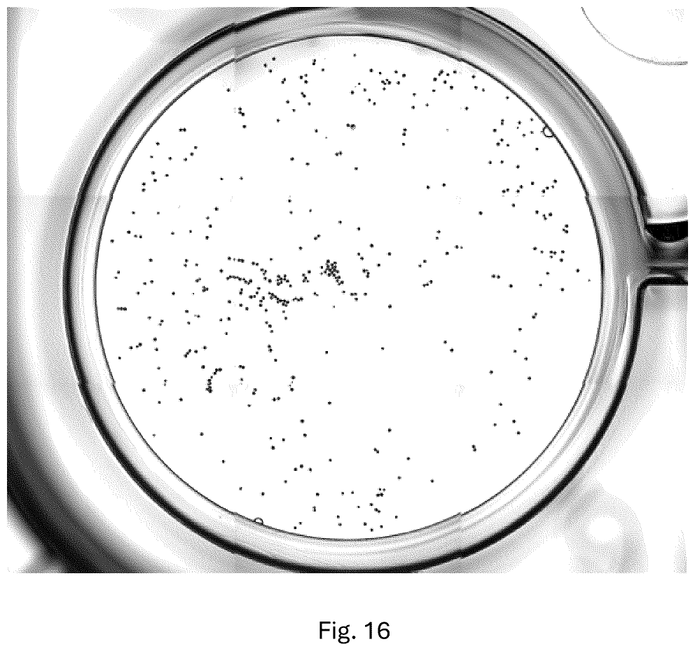

In addition to the method of counting pollen by flow cytometry (Amphasys) as described in Example 1 and other examples in this disclosure, other methods can also be used to count pollen grains. This example outlines the counting of pollen grains captured by stigmatic tissues using image analysis. This process provides pollen counts for pollen harvested and prepared by the same methods described in Example 1 and other examples herein, but quantifies the pollen in each sample using imaging software. The Echo Revolution microscope (ECHO, SanDiego, CA) captures images from up to 48 pollen samples simultaneously. For a 24-well plate of samples, this is accomplished by taking 3×3 images per well at 1.25× magnification and stitching the images together, which requires about 40 seconds per sample. Following image processing, pollen counts are generated using ImagePro analysis software. An example of a pollen sample used for imaging and counting is shown in FIG. 16.

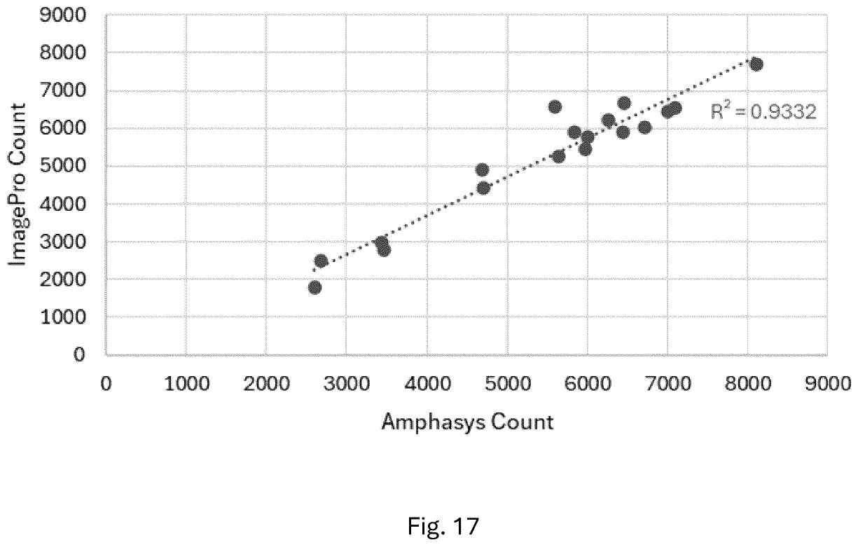

FIG. 17 shows a comparison of the counts obtained using flow cytometry (Amphasys) versus the microscope method. The x-axis shows the count using Amphasys, while the y-axis shows the count using the microscope and ImagePro analysis software. As shown in the figure, the counting capacity of the Echo Revolution microscope imaging and counting system compares extremely well (R2=0.9332) to the impedance flow cytometry method of Amphasys. In this case, pollen samples were collected from actively shedding maize tassels, filtered into 2 mL of Isotone, then divided evenly into two subsamples for analysis by the two methods. Each point represents an individual time point for pollen collection. The imaging method has the advantage of processing many samples simultaneously.

### Example 20: Measured Vs Predicted Kernel Per Ear Yield in Commercial Hybrid Seed Fields

The following experiment was conducted in eight commercial hybrid maize seed fields near Pekin, IL during July and August 2024. All eight fields were planted in a 4 female: 1 male row configuration typical of hybrid seed production. Whole silk bouquets were collected from 9 plants in each of the four female rows at five locations within each field prior to 50% silking. Pollen grains per bouquet were determined using the pollen counting system described in Examples 1 and 2, and quantified by Amphasys impedence flow cytometry. Kernels per ear were predicted from these values using the predictive pollen presence and yield model described in Example 15. The average kernels produced per ear for each field were determined at kernel maturity on 4 ears per female row at same five field locations.

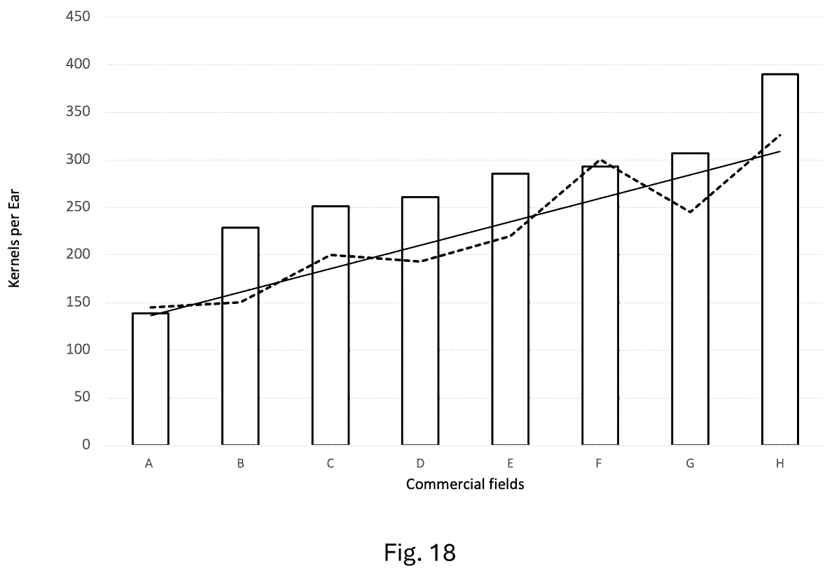

The bars in FIG. 18 show the average number of kernels per ear measured for each commercial field measured at harvest maturity. These average values varied from about 140 to about 390 kernels per ear across the eight commercial fields. This variation reflected both the asynchrony in male and female inbred reproductive development and the genetic potential for pollen production of the male inbreds and potential for kernel formation of the female inbreds as determined by in-field observations. The dashed line (——) on FIG. 18 shows the predicted kernel per ear values for each field based on the pollen on silk counts taken early in the flowering period. These measured and predicted values were closely correlated (y=24.631x+111.54, R²=0.8531). This remarkable result indicates the invention described herein provides commercially relevant information about the yield potential of a hybrid seed field midway through the production season. This information is of great value to the seed industry as it provides a forecast of product availability well in advance of harvest.

### Example 21: Prediction of Yield Response to Intentional Pollination

This experiment was conducted near Pekin, IL in a commercial hybrid maize seed production field in July and August 2024. The fields were planted in a 4 female: 1 male row configuration typical of hybrid seed production. As the population of female plants approached 50% silking (i.e., 50% of the plants had silks emerging from the enclosing ear husks), whole silk bouquets were collected from 9 plants in each of the four female rows at five locations within each field. Pollen counts per bouquet were determined using the pollen counting system described in Examples 1 and 2, and quantified by Amphasys impedence flow cytometry. Based on the pollen on stigma counts, the decision was made to apply an intentional pollination to supplement natural pollination. The intentional pollination used previously collected and preserved pollen and was applied to a portion of the field with the intention of increasing seed set. Pollen was collected mechanically and preserved for 2 days prior to mechanical application.

Two pollen application protocols were used. The first protocol was the intentional application of 2 liters of preserved pollen per acre, mechanically applied to all four female plant rows. The second protocol was the intentional application of 2 liters of preserved pollen per acre, but mechanically applied only to the two rows within the female plant block having the lowest pollen counts. In both cases, intentional pollinations were made 2 to 3 days after the female plant reached 50% silking. Resulting kernels per ear were predicted using the predictive pollen presence and yield model described in Example 15. The actual number of kernels produced per ear for each field were determined at kernel maturity on 4 ears per female row at the same five field locations.

Table 6 shows the pollen counts per silk bouquet during flowering, the predicted kernels per ear based on these counts, and the measured kernels per ear obtained at harvest maturity. Under natural pollination, plants harvested from the four female plant rows produced an average of 275 kernels per ear. The predicted value for these natural pollinations was 249 kernels per ear based on the stigmatic pollen counts taken during flowering. Intentionally pollinating all four female rows increased kernels per ear dramatically, with each ear gaining an average of 44 kernels. The predicted increase value also was dramatic at an average of 36 kernels. On a percentage basis, in this field, the predicted gain over natural pollination in response to the intentional pollination was nearly identical to the actual gain.

A similar trend was observed when only the two middle rows of the 4-row female plant block were intentionally pollinated. This approach was taken to evaluate whether a yield benefit would still occur if the preserved pollen supply were limited and could not be applied to all plants in each female block, or if female rows adjacent to the male pollen donor rows were receiving adequate pollen. Table 6 again shows a substantial benefit from the supplemental intentional pollination, which added an average of 66 additional kernels to the ears in the middle two rows. The predicted increase in kernels per ear was 46. The percent gain was about 19% and 26% over natural pollination for predicted and measured values, respectively. These novel and remarkable results indicate the invention described herein can be used to overcome the negative impacts of reproductive asynchrony by identifying fields in need of an intentional application of pollen which synchronously pollinates the exposed and receptive florets throughout the female plant population. It enables commercial seed growers to predict the benefit of such an intentional pollination intervention on expected seed yield. Furthermore, these results provide evidence that seed producers can select which rows are most in need of supplemental pollen to maximize ROI.

TABLE 6
Predicted and measured kernels per ear in a maize hybrid seed field
receiving a supplemental intentional pollination.

| | | Illinois 2024 | | | |
|---|---|---|---|---|---|
| | Row | K/ear | K/ear Natural | Gain | Gain |
| Field Evaluation | Evaluation | Natural | Plus Intentional | K/ear | % |
| Predicted by Pollen | All 4 rows | 249 | 285 | 36 | 14.5% |
| Measured 30 DAP | | 276 | 320 | 44 | 15.9% |
| Predicted by Pollen | Middle 2 | 240 | 286 | 46 | 19.2% |
| Measured 30 DAP | rows only | 250 | 316 | 66 | 26.4% |

1. A method of quantifying pollination efficiency, said method comprising:
a. growing a parent plant;
b. waiting for the stigmatic surfaces of said parent plant to become receptive and exposed to pollen;
c. intentionally pollinating said stigmatic surfaces with designated plant pollen collected from a pollen parent plant;
d. following pollen hydration and the entry of the pollen tube into the stigma, removing said pollinated stigmatic surfaces of said parent plant;
e. counting the number of pollen grains retained on the removed stigmatic surfaces of said parent plant, and
f. calculating the pollination efficiency for the intentional pollination.

2. The method of claim 1, further comprising predicting the parent plant's seed or fruit set based on the pollination efficiency calculation.

3. The method of claim 1, wherein the calculated pollination efficiency is used in one or more of the following evaluations:
a. An evaluation of the efficiency or efficacy of an intentional pollination method;
b. An evaluation of the efficiency or efficacy of equipment used to conduct intentional pollinations;
c. An evaluation to determine how much additional pollen should be intentionally applied to a plurality of plants in order to maximize seed or fruit set;
d. An evaluation to determine whether less pollen can be used in intentional pollination to successfully maximize seed or fruit set;
e. An evaluation of a treatment used on a plant wherein the treatment is intended to enhance successful pollination;
f. An evaluation of a treatment used on pollen to be intentionally applied to a plant wherein the treatment is intended to enhance pollen viability;
g. An evaluation of a treatment used on pollen to be intentionally applied to a plant wherein the treatment is intended to enhance or reduce pollen adhesion to stigmatic surfaces; and
h. An evaluation of a treatment used on pollen to be intentionally applied to a plant wherein the treatment is intended to increase pollination success.

4. The method of claim 1, further comprising the following steps:
a. determining whether the predicted seed or fruit set is near maximum seed or fruit set for the given plant species;
b. if the predicted seed or fruit set is below maximum, determining the necessary pollen grains/stigma dosage rate to achieve maximum seed or fruit set; and
c. conducting one or more intentional pollinations at the calculated dosage rate to achieve maximum seed or fruit set.

5. The method of claim 1, further comprising the following steps:
a. allowing the parent plant to mature and produce seed or fruit,
b. collecting the seed or fruit,
c. counting the seed or fruit, and
d. calculating the seed per pollen grain or fruit per pollen grain ratio.

6. The method of claim 1, wherein the removal of said pollinated stigmatic surfaces from said parent plant does not impact the successful fertilization of the plant ovule.

7. The method of claim 1, wherein the removal of said pollinated stigmatic surfaces from said parent plant occurs during a time period from 15 minutes following said intentional pollination to 7 days following said intentional pollination.

8. The method of claim 1, wherein all stigmatic surfaces are removed from said parent plant.

9. The method of claim 1 wherein the designated pollen used for intentional pollination is comprised of one or more of the following:
a. Fresh pollen
b. Preserved pollen.

10. The method of claim 1 wherein said designated pollen used in intentional pollinations is applied at the time which the parent plant first becomes receptive to said pollen.

11. The method of claim 1 wherein said designated pollen used in intentional pollinations is obtained from a single genetic source.

12. The method of claim 1 wherein said designated pollen used in intentional pollinations is obtained from multiple genetic sources and is combined prior to application.

13. The method of claim 1 wherein said designated pollen used in intentional pollinations is collected from a plant from a different genetic background to the parent plant.

14. The method of claim 1 wherein said designated pollen used in intentional pollinations is collected from a plant from a similar genetic background to the parent plant.

15. The method of claim 1 wherein said pollinations are conducted on parent plants intended for at least one of the following purposes:
a. seed production purposes,
b. grain production purposes, and
c. fruit production purposes.

16. The method of claim 1, wherein the method used to count pollen grains is selected from the group consisting of impedance flow cytometry, densitometry, fluorescence imaging, digital imaging, cytometry, spectrophotometry, densitometry, vital dyes, hemocytometry, and brightfield microscopy.

17. A method of quantifying pollination efficiency, said method comprising:
a. growing a plurality of plants;
b. waiting for the stigmatic surfaces of at least a portion of said plants to become receptive and exposed to pollen;
c. allowing said stigmatic surfaces to be naturally pollinated;
d. following said pollination, but within a period of stigmatic receptivity, removing said pollinated stigmatic surfaces of at least one of said plants;
e. counting the number of pollen grains retained on said removed pollinated stigmatic surfaces of said plant, and
f. calculating the pollination efficiency for the natural pollination of the plurality of plants.

18. The method of claim 17, further comprising predicting the parent plant's seed or fruit set based on the pollination efficiency calculation.

19. The method of claim 17, wherein the calculated pollination efficiency is used in one or more of the following evaluations:
a. An evaluation to determine how much additional pollen should be intentionally applied to a plurality of plants in order to maximize seed or fruit set;
b. An evaluation of a treatment used on a plant wherein the treatment is intended to enhance successful pollination.

20. The method of claim 17, further comprising the following steps:
a. determining whether the predicted seed or fruit set is near maximum seed or fruit set for the given plant species;
b. if the predicted seed or fruit set is below maximum, determining the necessary pollen grains per stigma dosage rate to achieve maximum seed or fruit set; and
c. conducting one or more intentional pollinations at the calculated dosage rate to achieve maximum seed or fruit set.

21. The method of claim 17, further comprising the following steps:
a. allowing the parent plant to mature and produce seed or fruit,
b. collecting the seed or fruit,
c. counting the seed or fruit, and
d. calculating the seed per pollen grain or fruit per pollen grain ratio.

22. The method of claim 17, wherein the removal of said pollinated stigmatic surfaces from said parent plant does not impact the successful fertilization of the plant ovule.

23. The method of claim 17, wherein the removal of said pollinated stigmatic surfaces from said parent plant occurs during a time period from 15 minutes following said natural pollination to 7 days following said natural pollination.

24. The method of claim 17, wherein all stigmatic surfaces are removed from said parent plant.

25. The method of claim 17, wherein the method used to count pollen grains is selected from the group consisting of impedance flow cytometry, densitometry, fluorescence imaging, digital imaging, cytometry, spectrophotometry, densitometry, vital dyes, hemocytometry, and brightfield microscopy.

26. A method of quantifying pollination efficiency, said method comprising:
a. growing a plurality of plants;
b. waiting for the stigmatic surfaces of at least a portion of said plants to become receptive and exposed to pollen;
c. allowing said stigmatic surfaces to be naturally pollinated;
d. following said natural pollination, intentionally pollinating said stigmatic surfaces with designated plant pollen;
e. following both the natural and intentional pollinations, but within a period of stigmatic receptivity, removing said pollinated stigmatic surfaces of at least one of said plants;
f. counting the number of pollen grains retained on said pollinated stigmatic surfaces of said plant, and
g. calculating the pollination efficiency for the combined natural and intentional pollination of the plurality of plants.

27. The method of claim 26, further comprising predicting the seed or fruit set from the plurality of plants based on the pollination efficiency calculation.

28. The method of claim 26, wherein the calculated pollination efficiency is used in one or more of the following evaluations:
a. An evaluation of the efficiency or efficacy of an intentional pollination method;
b. An evaluation of the efficiency or efficacy of equipment used to conduct intentional pollinations;
c. An evaluation to determine how much additional pollen should be intentionally applied to a plurality of plants in order to maximize seed or fruit set;
d. An evaluation to determine whether less pollen can be used in intentional pollination to successfully maximize seed or fruit set;
e. An evaluation of a treatment used on a plant wherein the treatment is intended to enhance successful pollination;
f. An evaluation of a treatment used on pollen to be intentionally applied to a plant wherein the treatment is intended to enhance pollen viability;
g. An evaluation of a treatment used on pollen to be intentionally applied to a plant wherein the treatment is intended to enhance or reduce pollen adhesion to stigmatic surfaces; and
h. An evaluation of a treatment used on pollen to be intentionally applied to a plant wherein the treatment is intended to increase pollination success.

29. The method of claim 26, further comprising the following steps:
a. determining whether the predicted seed or fruit set is near maximum seed or fruit set for the given plant species;
b. if the predicted seed or fruit set is below maximum, determining the necessary pollen grains per stigma dosage rate to achieve maximum seed or fruit set; and
c. conducting one or more intentional pollinations at the calculated dosage rate to achieve maximum seed or fruit set.

30. The method of claim 26, further comprising the following steps:
a. allowing the plurality of plants to mature and produce seed or fruit,
b. collecting the seed or fruit from one or more plants from the plurality,
c. counting the seed or fruit, and
d. calculating the seed per pollen grain or fruit per pollen grain ratio.

31. The method of claim 26, wherein the removal of said pollinated stigmatic surfaces from said plant does not impact the successful fertilization of the plant ovule.

32. The method of claim 26, wherein the removal of said pollinated stigmatic surfaces from said plant occurs during a time period from 15 minutes following said intentional pollination to 7 days following said intentional pollination.

33. The method of claim 26, wherein all stigmatic surfaces are removed from said plant.

34. The method of claim 26 wherein the designated pollen used for intentional pollination is comprised of one or more of the following:
a. Fresh pollen
b. Preserved pollen.

35. The method of claim 26 wherein said designated pollen used in intentional pollinations is applied at the time which the parent plant first becomes receptive to said pollen.

36. The method of claim 26 wherein said designated pollen used in intentional pollinations is obtained from a single genetic source.

37. The method of claim 26 wherein said designated pollen used in intentional pollinations is obtained from multiple genetic sources and is combined prior to application.

38. The method of claim 26 wherein said designated pollen used in intentional pollinations is collected from a plant from a different genetic background to the parent plant.

39. The method of claim 26 wherein said designated pollen used in intentional pollinations is collected from a plant from a similar genetic background to the parent plant.

40. The method of claim 26 wherein said pollinations are conducted on parent plants intended for at least one of the following purposes:
a. seed production purposes,
b. grain production purposes, and
c. fruit production purposes.

41. The method of claim 26 wherein the method used to count pollen grains is selected from the group consisting of impedance flow cytometry, densitometry, fluorescence imaging, digital imaging, cytometry, spectrophotometry, densitometry, vital dyes, hemocytometry, and brightfield microscopy.

42. A method of quantifying contaminating pollen presence, said method comprising:
a. growing a parent plant;
b. waiting for the stigmatic surfaces of said plant to become receptive and exposed to pollen;
c. before any desirable pollen in the growing environment begins to shed, allowing the stigmatic surfaces of said parent plant to be naturally pollinated;
d. at least 15 minutes following said natural pollination, removing said pollinated stigmatic surfaces of said parent plant;
e. counting the number of pollen grains retained on the said pollinated stigmatic surfaces of said plant, and
f. calculating the amount of contaminating pollen present.

43. A plurality of parent plants, wherein the parent plants have been grown until the stigmatic surfaces of said parent plants are receptive and exposed to pollen, and further wherein said stigmatic surfaces have been intentionally pollinated with pollen collected from a pollen parent plant, and wherein at least 15 minutes following said intentional pollination, said pollinated stigmatic surfaces of at least one of said parent plants has been removed, and the number of pollen grains retained on said pollinated stigmatic surfaces of said parent plant has been counted, such that the pollination efficiency for the intentional pollination of the plurality of parent plants can be calculated.

44. A pollinated stigmatic surface that has been removed from a parent plant in order to count the number of pollen grains that have been retained on said pollinated stigmatic surface in order to calculate the pollination efficiency.
